(19) United States
(12) Patent Application Publication
(10) Pub. No.: US 2011/0117008 A1
(43) Pub. Date: May 19, 2011

(54) USE OF AVICINS TO DELIVER THERAPEUTIC AND DIAGNOSTIC AGENTS

(75) Inventors: V. Prasad Shastri, Nashville, TN (US); Christopher Pino, Saline, MI (US); Jordan Gutterman, Houston, TX (US)

(73) Assignees: Research Development Foundation, Carson City, NV (US); Vanderbilt University, Nashville, TN (US)

(21) Appl. No.: 12/744,556

(22) PCT Filed: Nov. 24, 2008
(86) PCT No.: PCT/US2008/084523

§ 371 Date: Dec. 2, 2010

Related U.S. Application Data
(60) Provisional application No. 60/990,119, filed on Nov. 26, 2007.
(60) Provisional application No. 61/046,338, filed on Apr. 18, 2008.
(60) Provisional application No. 61/087,914, filed on Aug. 11, 2008.

(51) Int. Cl. A61K 51/00; A61K 31/704; C07H 15/256; A61K 49/00; A61P 35/00; A61P 9/00; A61P 7/02; A61P 23/00; A61P 29/00; A61P 31/00; A61P 31/12; A61P 43/00

(52) U.S. Cl. 424/1.11; 514/33; 536/17.9; 424/9.1

(57) ABSTRACT

The present invention provides compositions of avicins and avicin mixtures that enhance topical, percutaneous, transmucosal, transepithelial, transendothelial or transdermal transport of agents including diagnostic molecules, therapeutic drugs and cosmetic materials.

This application claims the benefit of priority to U.S. Provisional Application Ser. No. 60/990,119, filed Nov. 26, 2007, U.S. Provisional Application Ser. No. 61/046,338, filed Apr. 18, 2008, and U.S. Provisional Application Ser. No. 61/087,914, filed Aug. 11, 2008, the entire contents of all are hereby incorporated by reference.

### BACKGROUND OF THE INVENTION

1. Field of the Invention

The present invention relates generally to the field of medicine. More specifically, the invention relates to the use of avicin compositions to deliver diagnostic and therapeutic agents in percutaneous, transmucosal, transepithelial, transendothelial or transdermal modes.

2. Description of the Related Art

A variety of pharmaceuticals are currently delivered through the skin. This drug administration pathway is advantageous due to its delayed first pass metabolism, ease of application and high patient compliance, second only to oral administration in pill form. Oral medications, however, are susceptible to breakdown in the stomach, and may have limited bioavailability. Despite the promise of transdermal drug delivery, currently it is relegated to the administration of small molecule pharmaceuticals lower than 300 Daltons molecular weight. This size restriction is due to the structural characteristics of the lipids of the skin. The drugs normally chosen to be administered transdermally are hydrophobic, non-charged molecules. Water-soluble molecules rarely exhibit percutaneous absorption or transdermal transport when delivered alone, leaving major limitations with respect to drugs that may be delivered transdermally.

In order to deliver drugs larger than 300 Daltons, mechanical stimulation methods such as sonication and electroporation have been used (Prausnitz et al., 1993, Boucaud et al., 2002). In addition, chemicals known to alter permeability of molecules across skin have been added to pharmaceutical formulations to enhance skin permeability. The enhancers currently used are all lipophilic or lipid-like agents, so there remains a need to identify new classes of enhancers (Karande et al., 2004). Despite the development of mechanical and chemical enhancers, the molecular weight cutoff of skin has not been substantially augmented (Finnin et al., 1999; Purdon et al., 2004), and thus a need remains for improved methods and compositions that permit the delivery of high molecule weight and/or lipophobic agents.

### SUMMARY OF THE INVENTION

Thus, in accordance with the present invention, there is provided a pharmaceutical composition comprising (i) an avicin, and (ii) a therapeutic or diagnostic agent, wherein the composition is formulated for topical, percutaneous, transmucosal, transepithelial, transendothelial or transdermal administration. The avicin maybe Avicin D and/or Avicin G, and the composition may be essentially free of avicins other than Avicin D and/or Avicin G. The composition may also comprise at least one avicin in addition to Avicin D and Avicin G. In particular, the present invention contemplates an avicin mixture designated F094. The therapeutic agent may be an anti-cancer agent, such as a chemotherapeutic, a radioisotope, a toxin, a hormone, a cell-cycle regulator, a tumor suppressor, an anti-angiogenic agent, gene silencing agent, or a pro-apoptotic protein. The therapeutic agent may be an agent other than an anti-cancer agent, such as an antibiotic, an antiviral, an anti-inflammatory, a cardiovascular drug, a cosmeceutical, an anesthetic such as a water-soluble or local anesthetic (e.g., a lidocaine, a bupivacaine, or a prilocaine), a toxin, an anti-coagulant or a hormone. The diagnostic agent may be a protein, peptide or small molecule. The therapeutic or diagnostic agent may be hydrophilic or hydrophobic.

The composition may be formulated for topical administration, for example, in a cream, ointment, salve, spray, gel, lotion or emulsion. The composition may be formulated for transmucosal, transepithelial, transendothelial or transdermal administration. One example of transdermal formulation is a patch. The composition may also comprise a powder, liquid aerosol, or powder aerosol for use in topical pulmonary delivery and inhalation therapies. The composition may further comprise a chemical penetration enhancer, a membrane permeability agent, a membrane transport agent, a preservative, a surfactant or a stabilizer.

In another embodiment, there is provided a pharmaceutical composition comprising purified Avicin G free of other avicins, wherein the composition is formulated for topical or transdermal administration. The composition may further comprise a cosmeceutical agent, a cream, gel, salve, ointment lotion, emulsion or spray.

In still another embodiment, there is provided a method of promoting percutaneous, transmucosal, transepithelial, transendothelial or transdermal transport of a therapeutic or diagnostic agent in an animal subject comprising applying an avicin preparation and the agent to body surface. The animal subject may be a mammal, a human, a dog, a cat, a cow, a horse, a monkey, or a rabbit. The therapeutic agent may be an anti-cancer agent, such as a chemotherapeutic, a radioisotope, a toxin, a hormone, a cell-cycle regulator, a tumor suppressor, anti-angiogenic agent, gene silencing agent, or a pro-apoptotic protein. The therapeutic agent may be an agent other than an anti-cancer agent, such as an antibiotic, an antiviral, an anti-inflammatory, a cardiovascular drug, a cosmeceutical, an anesthetic such as a water-soluble or local anesthetic(e.g., a lidocaine, a bupivacaine, or a prilocaine), a toxin, an anti-coagulant or a hormone. The diagnostic agent may be a protein, peptide or small molecule. The therapeutic or diagnostic agent may be hydrophilic or hydrophobic.

The composition may be formulated for topical administration, for example, in a cream, ointment, salve, spray, gel, lotion or emulsion. The composition may be formulated for transmucosal, transepithelial, transendothelial or transdermal administration. One example of transdermal formulation is a patch. The composition may also comprise a powder, liquid aerosol, or powder aerosol for use in topical pulmonary delivery and inhalation therapies. The composition may further comprise a chemical penetration enhancer, a membrane permeability agent, a membrane transport agent, a preservative, a surfactant or a stabilizer.

In yet another embodiment, there is provided a method of altering water retention by a tissue comprising identifying a human subject in need of water retention alteration, and applying a pharmaceutical formulation comprising an avicin to a tissue surface. In particular, Avicin D may be utilized to enhance water transport across a tissue surface, such as skin, while Avicin G may be utilized to inhibit water transport across a surface.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Structures for Avicins D and G.

FIG. 2—F904 increases skin permeability to water. In an experiment to assess the affect of F094 on water permeability of skin, donor compartment solutions of PBS with 0 mg/ml (control) and 10 mg/ml of F094 were used. In addition, it was desired to access if the known enhancer N-Methyl Pyrrolidinone (NMP) might modulate skin's water permeability when codelivered. Ten percent NMP was used as a “NMP control”, and a solution of 10% NMP and 10 mg/ml F094 was used to test codelivery. Each donor compartment solution was loaded with 5 μCi/ml (1 μg/ml) of tritiated (³H) water. Receiver compartments were sampled at 0.5, 1, 2, 3, 6, 12, 18 and 24 hours.

FIG. 3—F904 (20 mg/ml) increases skin permeability to estradiol. In an experiment to assess the affect of F094 on estradiol permeability of skin, donor compartment solutions of PBS with 0 mg/ml (control), 5 mg/ml, 10 mg/ml and 20 mg/ml of F094 were used. Each solution was loaded with 5 μCi/ml of 3H-Estradiol (Amersham TRK 322). Receiver compartments were sampled at 0.5 1, 2, 3, 6, 12, 18 and 24 hours.

FIG. 4—F904 improves estradiol localization in the skin. Using a mass balance equation, amount of percutaneous absorption could be calculated. Percutaneous Absorption=Starting amount in Donor−Final amount in Donor−Transported amount in Receiver.

FIG. 5—Avicin G transports across full thickness skin. F904 enhances delivery. In order to access if Avicins might be able to transport across skin, radiolabeled ¹²⁵I-Avicin G was applied on its own and also codelivered with increasing amounts of F094. Experimental groups all contained 5 μCi/ml of Avicin G with experimental groups of 0, 20, 40 and 80 mg/ml F094. Receiver compartments were sampled at 1, 2, 3, 6, 12, 18 and 24 hours.

FIG. 6—F904 improves Avicin G localization in the skin. Again, using a mass balance equation, amount of percutaneous absorption could be calculated. Percutaneous Absorption=Starting amount in Donor−Final amount in Donor−Transported amount in Receiver.

FIG. 7—Avicin D increases water permeability of skin as compared to Avicin G. Each donor compartment was loaded with 2 ml of total solution to hydrostatically balance with each receiver compartment. Experimental groups that were tested included: control, F094, Avicin D and Avicin G. The control solution consisted of PBS and tritiated water (1 μg/ml, 5 μCi/ml), however, no Avicin or F094 was added. The F094 solution was formulated by diluting stock F094 solution into PBS (10 mg/ml), also containing tritiated water (1 μg/ml, 5 μCi/ml) for transport. Likewise, Avicin D and Avicin G solutions contained 1 mg/ml of Avicin D and G respectively, along with PBS and tritiated water (1 μg/ml, 5 μCi/ml). Receiver compartments were sampled at 1, 2, 3, 6, 12, 18 and 24 hours.

FIG. 8—Avicin D increase estradiol permeability of skin as compared to Avicin G. Each donor compartment was loaded with 2 ml of total solution to hydrostatically balance with each receiver compartment. Experimental groups that were tested included: control, F094, Avicin D and Avicin G. The control solution consisted of PBS and 3H-estradiol (5 μCi/ml), however, no Avicin or F094 was added. The F094 solution was formulated by diluting stock F094 solution into PBS (10 mg/ml), also containing 3H-estradiol (5 μCi/ml) for transport. Likewise, Avicin D and Avicin G solutions contained 1 mg/ml of Avicin D and G respectively, along with PBS and 3H-estradiol (5 μCi/ml). Receiver compartments were sampled at 1, 2, 3, 6, 12, 18 and 24 hours.

FIG. 9—Lidocaine HCl Transport Through Full Thickness Porcine Skin. In an experiment to assess the effect of F094 on Lidocaine HCl transport through skin, donor compartment solutions of PBS with 0 mg/ml (control), 1 mg/ml, 5 mg/ml, and 10 mg/ml of F094 were used. Receiver compartments were sampled at 0, 3, 6, 12, 18 and 24 hours. HPLC was used to quantify delivered mass, and to calculate permabilities in lag times. Error bar represents mean+/−standard deviation.

FIG. 10—Prilocaine HCl Transport Through Full Thickness Porcine Skin. In an experiment to assess the affect of F094 on Prilocaine HCl transport through skin, donor compartment solutions of PBS with 0 mg/ml (control), 1 mg/ml, 5 mg/ml, and 10 mg/ml of F094 were used. Receiver compartments were sampled at 0, 3, 6, 12, 18 and 24 hours. HPLC was used to quantify delivered mass, and to calculate permabilities in lag times. Error bar represents mean+/−standard deviation.

### DETAILED DESCRIPTION OF THE INVENTION

### I. THE PRESENT INVENTION

Avicin D and G are triterpene saponin glycosides that can be extracted from a desert plant, Acacia victoriae. Avicins have known anti-tumorigenic, anti-oxidant, and anti-inflammatory properties that have been studied in depth (Haridas et al., 2001; Haridas et al., 2003; Haridas et al., 2004; Gutterman et al., 2005; Haridas et al., 2007). However, because of their high molecular weight (2,000 Daltons; Jayatilake et al., 2003), it would be expected that these molecules would not exhibit percutaneous absorption or transdermal transport.

As shown herein, the inventors have found that not only do avicins exhibit percutaneous absorption and transdermal delivery, but they also exhibit enhancing effects on the delivery of both water and lipophillic drugs. This result is counterintuitive given what was previously known about avicins. Thus, it is now proposed that formulations with avicins as enhancers have the potential to vastly expand the types of drugs that can be administered transdermally, both in terms of the size of such drugs and also those with previously unfavorable hydrophilic/lipophilic balance (HLB) and charge attributes. As such, avicins as percutaneous/transdermal delivery enhancers have wide-ranging and significant applications in both the medical and cosmetic industries.

In the context of the present invention, transdermal delivery is defined as delivery that is localized in the subcutaneous/epidermal region, but is accumulated in the dermis, and as such will result in introduction of the agent into systemic circulation through the capillary bed. Topical delivery is defined herein as limited predominantly to the subcutaneous/epidermal region. Percutaneous delivery would entail adsorption beyond the subcutaneous region.

The details of the invention are explained further, below.

### II. AVICINS

A. General Information

Triterpenoids form the largest and most diverse class of organic compounds found in plants (Mahato & Sen, 1997). They exhibit enormous chemical variety and complexity but have a common biosynthetic origin, the fusion of five-carbon units, each having an isoprenoid structure (Wendt et al., 2000). Methods for isolating, characterizing, modifying, and using triterpenoid compounds can be found in U.S. Pat. No. 6,444,233, which is incorporated in its entirety by reference.

Triterpene saponins particularly have been the subject of much interest because of their biological properties. Pharmacological and biological properties of triterpene saponins from different plant species have been studied, including fungicidal, anti-viral, anti-mutagenic, spermicidal or contraceptive, cardiovascular, and anti-inflammatory activities (Hostettmann et al., 1995).

Triterpenoids that exhibit pharmacological properties include glycyrrhetinic acid, and certain derivatives thereof, which are known to have anti-ulcer, anti-inflammatory, anti-allergic, anti-hepatitis and antiviral actions. For instance, certain glycyrrhetinic acid derivatives can prevent or heal gastric ulcers (Doll et al., 1962). Among such compounds known in the art are carbenoxolone (U.S. Pat. No. 3,070,623), glycyrrhetinic acid ester derivatives having substituents at the 3° position (U.S. Pat. No. 3,070,624), amino acid salts of glycyrrhetinic acid (Japanese Patent Publication JP-A-44-32798), amide derivatives of glycyrrhetinic acid (Belgian Patent 753773), and amide derivatives of 11-deoxoglycyrrhetinic acid (British Patent 1346871). Glycyrrhetinic acid has been shown to inhibit enzymes involved in leukotriene biosynthesis, including 5-lipoxygenase activity, and this is thought to be responsible for the reported anti-inflammatory activity (Inoue et al., 1986).

Betulinic acid, a pentacyclic triterpene, is reported to be a selective inhibitor of human melanoma tumor growth in nude mouse xenograft models and was shown to cause cytotoxicity by inducing apoptosis (Pisha et al., 1995). A triterpene saponin from a Chinese medicinal plant in the Cucurbitaceae family has demonstrated anti-tumor activity (Kong et al., 1993). Monoglycosides of triterpenes have been shown to exhibit potent and selective cytotoxicity against MOLT-4 human leukemia cells (Kasiwada et al., 1992) and certain triterpene glycosides of the Iridaceae family inhibited the growth of tumors and increased the life span of mice implanted with Ehrlich ascites carcinoma (Nagamoto et al., 1988). A saponin preparation from the plant Dolichos falcatus, which belongs to the Leguminosae family, has been reported to be effective against sarcoma-37 cells in vitro and in vivo (Huang et al., 1982). Soya saponin, also from the Leguminosae family, has been shown to be effective against a number of tumors (Tomas-Barbaren et al., 1988). Some triterpene aglycones also have been demonstrated to have cytotoxic or cytostatic properties, i.e., stem bark from the plant Crossopteryx febrifuga (Rubiaceae) was shown to be cytostatic against Co-115 human colon carcinoma cell line in the ng/ml range (Tomas-Barbaren et al., 1988).

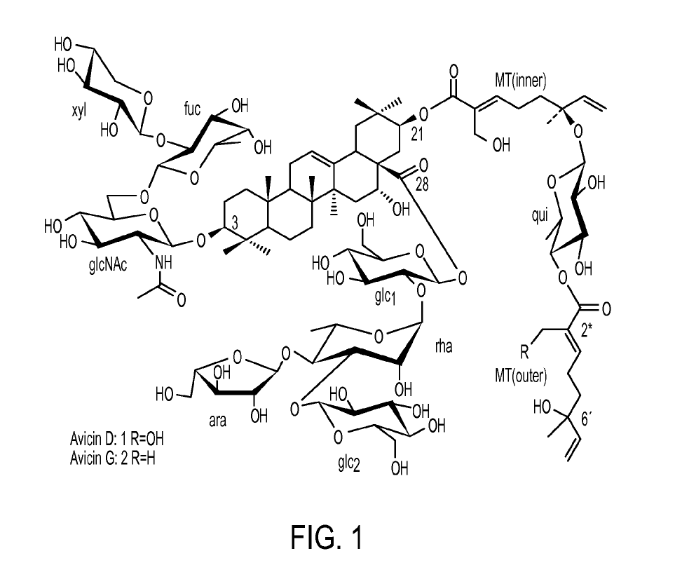

Avicins are triterpenoid electrophilic metabolite molecules isolated from an Australian desert plant, Acacia victoriae. A series of studies have identified cancer and inflammatory diseases as potential clinical targets for avicins (Haridas et al., 2001; Haridas et al., 2001; Haridas et al., 2004; Hanausek et al., 2001; Mujoo et al., 2001; Jayatilake et al., 2003). There is evidence that avicins induce stress resistance in human cells in a redox dependent manner, and that their pro-apoptotic property appears to be independent of p53. In particular, the present invention contemplates the use of Avicins D and G, mixtures thereof, as well as mixtures of Avicins D and/or G with other avicins. The structures for Avicins D and G are shown in FIG. 1.

B. Purification

The present invention contemplates the use of a variety of different avicin preparations. In particular, Avicin D may be prepared as a crude mixture, a crude fraction with enhanced Avicin D content over a natural composition, or either a partially purified, essentially pure or purified to homogeneity Avicin D compositions. In addition, Avicin G may be prepared as a crude mixture, a crude fraction with enhanced Avicin G content over a natural composition, or either a partially purified, essentially pure or purified to homogeneity Avicin G compositions. Purification of Avicin D or G may be such that the composition is 10%, 20%, 30%, 40%, 50%, 60%, 70%, 80%, 90%, 95%, 98%, 99% or 100% Avicin D, Avicin G or Avicin D+G. In particular, an avicin composition comprising both Avicin D and G has been described, and is designated at fraction 94, or F094.

Purification of Avicin D, G and F094 is described in Jayatilake et al. (2003). Briefly, preparative HPLC separations were performed on a NovaPrep 800 system. Analytical HPLC separations (column: Intersil RP18, 4.6 150 mm) with a CH3CN—H2O solvent system (30-42% in 42 min, flow rate 1.0 ml/min) were carried out on a Hitachi D-6000 instrument equipped with a diode array detector. The detector in the HPLC systems was set at 220 nm. TFA (0.1%) was added to H2O used in the purification and analytical steps. Ground seed pods of A. victoriae (2.5 kg) were extracted in 20% aqueous MeOH (40 L) at 50° C. for 20 h. The dried extract (614 g) was fractionated on a Biotage-75 radial compression module with a C18 cartridge (7.5 30 cm) system in increments of 150-200 g of extract per run. The column was eluted with increasing amounts of MeOH—H2O (50%; 11 L, 60%, 65%, 70%, 75%, 80%; each 5 L). The fractions were analyzed by HPLC, and the 75% aqueous MeOH eluate, with F094 (90 g) being selected for further fractionation based on the observed bioactivity. The F094 extract (70 g) was refractionated (500 mL fractions) over the same Biotage cartridge with the MeOH—H2O solvent system (60%; 5 L, 70%; 13 L, 75%; 15 L, 80%; 2 L) to give two combined fractions (D-pool, 27.3 g; G-pool, 11.4 g). The D-pool in 1.0 g aliquots was separated by preparative HPLC (Flurosep-RP phenyl column, 50 250 mm, 10 ím, flow rate 80 mL/min) with 28% aqueous CH3CN. The avicin D-rich fractions in 100 mg aliquots were further purified on the same preparative HPLC column with 56% aqueous MeOH to give avicin D (1, 1.1 g). The G-pool was similarly subjected to repeated preparative HPLC purification employing as solvent systems 61% aqueous MeOH followed by 30% aqueous CH3CN to furnish avicin G (2, 0.35 g).

### III. DIAGNOSTIC AGENTS AND USES THEREFOR

A. Agents

The present invention may find use in the transport of various diagnostic agents. A variety of different agents are contemplated as “diagnostics” including nucleic acids (oliogo- and polynucleotides), proteins (including antibodies and other ligand-binding proteins), peptides, lipids, carbohydrate and small molecules. In general, thought not necessarily, such diagnostics will comprise a detectable label such as a fluorescent label, a radiolabel, a chemilluminscent label, a dye or a pigment.

Particular radiolabels used for diagnostics include carbon-11, nitrogen-13, oxygen-15 and fluorine-18 (positron emitters used in PET for studying brain physiology and pathology), cobalt-57 (a marker to estimate organ size), gallium-67 (tumor imaging and localisation of inflammatory lesions), indium-111 (for specialist diagnostic studies, e.g., brain studies, infection and colon transit studies), iodine-123 (used for diagnosis of thyroid function), krypton-81m from Rubidium-81 (imaging of pulmonary ventilation, e.g., in asthmatic patients, and for the early diagnosis of lung diseases and function), rubidium-82 (PET agent in myocardial perfusion imaging), strontium-92 (‘parent’ in a generator to produce Rb-82), and thallium-201 (diagnosis of coronary artery disease other heart conditions).

B. Uses

The avicin compositions of the present invention can be used advantangeously in a variety of diagnostic settings. For example, due to likely preferential partitioning in tumors, tumor imaging using MRI, CT and PET scanning technologies are contemplated to benefit from the use of avicins linked to various imaging agents. In addition, the avicins may be used in the context of known targeting vehicles such as liposomes, lipids, microparticles, and nanoparticles to enhance the penetration of imaging agents included therein.

### IV. THERAPEUTIC/COSMETIC AGENTS

The present invention contemplates the use of various therapeutic/cosmetic agents in conjunction with an avicin composition comprising Avicin D, Avicin G and mixtures thereof. The agents may be of widely varying nature and function, and used for a wide variety of indications, as set forth below.

A. Therapeutic Agents

(i) Anti-Cancer Agents

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

1. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. Alkylating agents can be implemented to treat chronic leukemia, non-Hodgkin's lymphoma, Hodgkin's disease, multiple myeloma, and particular cancers of the breast, lung, and ovary. They include: busulfan, chlorambucil, cisplatin, cyclophosphamide (cytoxan), dacarbazine, ifosfamide, mechlorethamine (mustargen), and melphalan. Troglitazaone can be used to treat cancer in combination with any one or more of these alkylating agents, some of which are discussed below.

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

Chlorambucil (also known as leukeran) is a bifunctional alkylating agent of the nitrogen mustard type that has been found active against selected human neoplastic diseases. Chlorambucil is known chemically as 4-[bis(2-chlorethyl)amino]benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remington's Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation. Thus, it can be used in combination with troglitazone in the treatment of cancer.

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 m g/m², 1.0 mg/m², 1.50 mg/m², 1.75 mg/m², 2.0 mg/m², 3.0 mg/m², 4.0 mg/m², 5.0 mg/m² , 10 mg/m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl)phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other B-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

Melphalan, also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL-) form is known as merphalan or sarcolysin. Melphalan is insoluble in water and has a pKa₁ of ˜2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

2. Antimetabolites

Antimetabolites disrupt DNA and RNA synthesis. Unlike alkylating agents, they specifically influence the cell cycle during S phase. They have used to combat chronic leukemias in addition to tumors of breast, ovary and the gastrointestinal tract. Antimetabolites include 5-fluorouracil (5-FU), cytarabine (Ara-C), fludarabine, gemcitabine, and methotrexate.

5-Fluorouracil (5-FU) has the chemical name of 5-fluoro-2,4(1H,3H)-pyrimidinedione. Its mechanism of action is thought to be by blocking the methylation reaction of deoxyuridylic acid to thymidylic acid. Thus, 5-FU interferes with the syntheisis of deoxyribonucleic acid (DNA) and to a lesser extent inhibits the formation of ribonucleic acid (RNA). Since DNA and RNA are essential for cell division and proliferation, it is thought that the effect of 5-FU is to create a thymidine deficiency leading to cell death. Thus, the effect of 5-FU is found in cells that rapidly divide, a characteristic of metastatic cancers.

3. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Thus, they are widely used for a variety of cancers. Examples of antitumor antibiotics include bleomycin, dactinomycin, daunorubicin, doxorubicin (Adriamycin), and idarubicin, some of which are discussed in more detail below. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

Doxorubicin hydrochloride, 5,12-Naphthacenedione, (8s-cis)-10-[(3-amino-2,3,6-trideoxy-a-L-lyxo-hexopyranosyl)oxy]-7,8,9,10-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat-cell carcinoma, non-small cell lung carcinoma, gastric adenocarcinoma, retinoblastoma, neuroblastoma, mycosis fungoides, pancreatic. carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 m g/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

In the present invention the inventors have employed troglitazone as an exemplary chemotherapeutic agent to synergistically enhance the antineoplastic effects of the doxorubicin in the treatment of cancers. Those of skill in the art will be able to use the invention as exemplified potentiate the effects of doxorubicin in a range of different pre-cancer and cancers.

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-hydrochloride; also termed cerubidine and available from Wyeth. Daunorubicin intercalates into DNA, blocks DAN-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myelocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg, 20 mg, or 10 mg I.V., the maximal serum concentrations were 2.4 mg/ml, 1.7 mg/ml, and 0.52 mg/ml, respectively. Clearance is effected primarily by metabolism in the liver, but metabolism occurs in other tissues as well. The rate of clearance is inversely proportional to the maximal serum concentration because, it is thought, of saturation of the degradative pathways. Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar.

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors that fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0.5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

Bleomycin is a mixture of cytotoxic glycopeptide antibiotics isolated from a strain of Streptomyces verticillus. Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin. Bleomycin may be given by the intramuscular, intravenous, or subcutaneous routes. It is freely soluble in water.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

4. Mitotic Inhibitors

Mitotic inhibitors include plant alkaloids and other natural agents that can inhibit either protein synthesis required for cell division or mitosis. They operate during a specific phase during the cell cycle. Mitotic inhibitors comprise docetaxel, etoposide (VP16), paclitaxel, taxol, taxotere, vinblastine, vincristine, and vinorelbine.

VP16 is also known as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/m², routinely 35 mg/m², daily for 4 days, to 50 mg/m², daily for 5 days have also been used. When given orally, the dose should be doubled. Hence the doses for small cell lung carcinoma may be as high as 200-250 mg/m². The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

Taxol is an antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

Vinblastine is another example of a plant aklyloid that can be used in combination with troglitazone for the treatment of cancer and precancer. When cells are incubated with vinblastine, dissolution of the microtubules occurs.

Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours. Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 mg/m², 6 mg/m², 8 mg/m², 9 mg/m², 10 mg/m², 20 mg/m², can be given. Of course, all of these dosages are exemplary, and any dosage in-between these points is also expected to be of use in the invention.

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

Camptothecin is an alkaloid derived from the chinese tree Camptotheca acuminata Decne. Camptothecin and its derivatives are unique in their ability to inhibit DNA Topoisomerase by stabilizing a covalent reaction intermediate, termed “the cleavable complex,” which ultimately causes tumor cell death. It is widely believed that camptothecin analogs exhibited remarkable anti-tumour and anti-leukaemia activity. Application of camptothecin in clinic is limited due to serious side effects and poor water-solubility. At present, some camptothecin analogs (topotecan; irinotecan), either synthetic or semi-synthetic, have been applied to cancer therapy and have shown satisfactory clinical effects. The molecular formula for camptothecin is C₂₀H₁₆N₂O₄, with a molecular weight of 348.36. It is provided as a yellow powder, and may be solubilized to a clear yellow solution at 50 mg/ml in DMSO 1N sodium hydroxide. It is stable for at least two years if stored at 2-8° C. in a dry, airtight, light-resistant environment. A variant of camptothecin, 9-nitro-camptothecin is also contemplated as useful in many of the same embodmients as camptothecin itself.

5. Nitrosureas

Nitrosureas, like alkylating agents, inhibit DNA repair proteins. They are used to treat non-Hodgkin's lymphomas, multiple myeloma, malignant melanoma, in addition to brain tumors. Examples include carmustine and lomustine.

Carmustine (sterile carmustine) is one of the nitrosoureas used in the treatment of certain neoplastic diseases. It is 1,3-bis(2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 m g/m², 30 mg/m², 40 mg/m², 50 mg/m², 60 mg/m², 70 mg/m², 80 mg/m², 90 mg/m² or 100 mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject.

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per ml) and in absolute alcohol (70 mg per ml). Lomustine is relatively insoluble in water (<0.05 mg per ml). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours. The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

6. Radioisotopes

Radiation therapy used according to the present invention may include, but is not limited to, the use of γ-rays, X-rays, and/or the directed delivery of radioisotopes to tumor cells. Such isotopes (and their half-life) include molybdenum-99 (66 h), technetium-99m (6 h), bismuth-213 (46 min), chromium-51 (28 d), cobalt-60 (10.5 mth), copper-64 (13 h), dysprosium-165 (2 h), erbium-169 (9.4 d), holmium-166 (26 h), iodine-125 (60 d), iodine-131 (8 d), iridium-192 (74 d), iron-59 (46 d), lutetium-177 (6.7 d), palladium-103 (17 d), phosphorus-32 (14 d), potassium-42 (12 h), rhenium-186 (3.8 d), rhenium-188 (17 h), samarium-153 (47 h), selenium-75 (120 d), sodium-24 (15 h), strontium-89 (50 d), xenon-133 (5 d), ytterbium-169 (32 d), ytterbium-177 (1.9 h), yttrium-90 (64 h), and radioisotopes of cesium, gold and ruthenium.

7. Other Anti-Cancer Agents

Other agents that may be used include Herceptin, Avastin, Iressa, Erbitux, Velcade, and Gleevec. In addition, growth factor inhibitors and small molecule kinase inhibitors have utility in the present invention as well. Gene therapies, using genes for tumor suppressors such as Rb, p53, mda-7, PTEN, BRCA-1, C-CAM, and regulators of programmed cell death (e.g., Bax, Bak, Bik, Bim, Bid, Bad, Harakiri) also can be utilized in both viral and non-viral expression vectors.

(ii) Anti-Microbial Agents

Classes of antibiotics that may be used in conjunction with compounds of the present invention include, but are not limited to, macrolides (e.g., erythromycin), penicillins (e.g., nafeillin), cephalosporins (e.g., cefazolin), carbepenems (e.g., imipenem, aztreonam), other beta-lactam antibiotics, beta-lactam inhibitors (e.g., sulbactam), oxalines (e.g., linezolid), ATP synthase inhibitors (e.g. diarylquinoline compounds, R207910), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, polyenes (e.g., amphotericin B), rifamycins (e.g., rifampin), and azoles (e.g., fluconazole).

Examples of specific antibiotics that may be used include, but are not limited to, nafcillin, methicillin, oxacillin, cloxacillin, dicloxacillin, ampicillin, amoxicillin, carbenicillin, ticarcillin, mezlocillin, piperacillin, erythromycin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, levofloxacin, grepafloxacin, norfloxacin, nalidixic acid, sparfloxacin, pefloxacin, amifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, R207910 and nystatin.

Anti-viral drugs include amantadine, rimantadine, pleconaril. nucleotide or nucleoside analogues that look like the building blocks of RNA or DNA (e.g, acyclovir, zidovudine, lamivudine), fomivirsen, morpholino oligos, ribozymes, protease inhibitors, zanamivir (Relenza®), oseltamivir (Tamiflu®), monolonal antibodies and interferons.

A variety of antifungal agents have been approved or are in development. These include the allylamines (amorolfine, butenafine, naftifine, terbinafine), anti-metabolites (flucytosine, fluconazole, intraconazole, ketoconazole, posaconazole, ravuconazole), azoles (voriconazole, clotrimazole, econazole, miconazole, oxiconazole, sulconazole, terconazole, tioconazole), chitin synthase inhibitors (nikkomycin Z, caspofungin), glucan synthase inhibitors (micafungin, anidulafungin, amphotericin B, AmB Lipid Complex, AmB Colloidal Dispersion), polyenes (Liposomal AmB, AmB oral suspesion, liposomal nystatin, topical nystatin, pimaricin), other systemics (griseofulvin, ciclopirox olamine) and other topicals (haloprogrin, tolnaftate, undecylenate). The use of drugs to treat fungal nail infections has particular relevance to the present invention.

(iii) Anti-Inflammatory Agents and Anesthetics/Analgesics

A wide variety of compounds find use in the reduction of pain or discomfort associated with disease, physical exertion, injury or therapies. Such compounds include lidocaine (e.g., lidocaine HCl), prilocaine (e.g., prilocaine HCL), bupivacaine (e.g., bupivacaine HCl), salicylates (choline & magnesium sulfates, diflunisal, salsalate, aspirin), NSAIDS (naproxen, flurbiprofen, diclofenac, sulindac, oxaprozin, piroxicam, indomethacin, etodolac, meloxicam, mefenamic acid, meclofenamate, ibuprofen, fenoprofen, ketoprofen, nabumetone, tolmetin, ketorolac), Celebrex®, Toradol®, steroids, opioids (morphine, oxycodone, levorphanol, methadone, codeine, oxymorphone, fentanyl, buprenorphine), propoxyphene, burtophanol tartrate, pentazocine, tramadol,

(iv) Hair Loss

A variety of agents may be delivered to the skin to address hair loss. Agents that promote blood flow, such as L-arginine and L-arg/HCl, as well as the drugs minoxidil and finasteride, are candidates for such delivery.

(v) Vaccine Compositions

The skin is a highly competent immunologic organ, and the epidermis contains cells that are dedicated to identifying and presenting foreign antigens to the host's defense system. As such, the skin constitutes an excellent target for vaccine delivery. Thus, vaccine compositions may be delivered using topical, transcutaneous or transdermal delivery approaches and may encode antigens such as viral, fungal, bacterial or parasite antigens or tumor antigens. Particular vaccines targeting dendritic cells include prostate cancer, lung cancer, and soft tissue sarcoma.

(vi) Hormones

Hormones for therapeutic use include corticosteroids, androgens, anabolic steroids, estrogen or estrogen derivatives, gonadotropins, insulin, sulfonylureas, parathyroid hormones, pituitary hormones (growth hormone), progestins, thyroid drugs, antithyroid drugs, and calcitonin all have been used to treat various conditions or diseases.

(vii) Cytokines, Growth Factors and Chemokines

Clinical uses for many cytokines, growth factors and chemokines have now been established. Cytokines include the interleukins (IL1α, IL1β, IL2, IL3, IL4, IL5, IL6, IL7, IL8, IL9, IL10, IL11, IL12, IL13, IL14, IL15, IL16, IL17, IL18, IL19, IL20, IL21, IL22, IL23, IL24, IL25, IL26) and interferons α, β and γ. Growth factors include EGF, PDGF, FGF (FGFR1-3), TGFβ, TGFα, Epo, IGF-II, TNFα, TNFβ and the colony stimulating factors (CSFs). Inteferon a and several of the interleukins (1, 2, 6 and 8) are also considered growth factors.

(viii) Gene/Nucleic Acid Delivery

Gene therapy delivery to and across the dermis has signficant application in a variety of disease states. Delivery usually relies on viral vectors to transfer and express genetic material, or on non-viral vectors which are encapsulated in lipid vehicles such as liposome and nanoparticles. Typical subjects for gene transfer are tumor suppressors, pro-apoptotic genes, regulators of cell cycle for cancer embodiments, enzymes and antigenic, immunostimulatory or immunosuppressive genes. Other nucleic acids can be delivered without use of expression constructs and include antisense, siRNAs, ribozymes, and miRNAs.

(ix) Cardiovascular Therapies

Another area of potential interest for transdermal delivery is in cardiovascular disease. A variety of transdermal preparations are presently available, including nitroglycerin (glyceryl nitrate), β-blockers (metoprolol, propranolol, atenolol, timolol, levobunolol, bopindolol, mepindolol, sotalol, labetolol, pindolol, acebutolol and oxprenolol), anti-coagulants, and calcium sensitizers (e.g., levosimendan).

(x) Respiratory Drugs

Respiratory ailments are amenable to delivery of drugs across tissue surfaces. For example, drugs such as β₂ agonists (peroral, periodical, retarded theophylline or peroral/retarded) may be delivered in a transdermal patch or in an inhaled composition to treat asthma or chromic obstructive pulmonary disease. Specific drugs include formoterol, salmeterol, salbutamol, terbutline, and tulobuterol.

(xi) Neurologic Drugs

A variety of different neurologic diseases and disorders can be treated with drugs, and the transdermal delivery of such drugs constitutes yet another embodiment of the present invention. Such diseases include Parkinson's (levodopa, dopamine agonists: bromocriptine, dihydroergocryptine, lisuride, pergolide, cabergoline, pramipexole, rotigotine; apomorphine, selegi line), depression (monoamine oxidase inhibitors: selegiline), bipolar disorder (lithium), restless leg syndrome (rotigotine), Alzheimer's (cholinesterase inhibitors: tacrine, donepezil, rivastigmine; muscarinic receptor antagonists: arecoline;), Tourette's syndrome (nicotine), ADHD (methylphenidate), tinnitius/vertigo (promethazine, meclizine, dextroamphetamine, procloperazine, diazepam), epilepsy (lidocaine, carbamazepine analogs, nicotine) and migraine (lidocaine, codeine, methylsergide maleate, calcium blockers, β blockers, tricyclic anti-depressants, aspirin, triptans: sumatriptan).

(xii) Musculoskeletal Disease Drugs

A variety of musculoskeletal diseases are presently amenable to treatment. For example, osteoporosis are osteoarthritis highly drugable diseases. Hormone replacement therapy (estrogen), vitamine D and calcium supplementation all are used to address osteoporosis, while osteoarthritis generally treateed with anti-inflammatories.

(xiii) Diabetes

Transdermal delivery of muslin to treat diabetes has been greatly enhanced by the development of genetically engineered human monocompetent insulin. A variety of different transdermal systems are currently in development.

(xiv) Sexual Disorders

Male sexual dysfunction, primarily erectile dysfunction, is currently being treated with topical formulations, including alprotadil (synthetic prostaglandin E1) and phosphodiesterase-5 inhibitors (sildenafil, vardenafil, tadalafil). Hypogonadism is treated with hormonal therapy (androgens such as testosterone).

Female sexual arousal disorder (FSDA) has no currently approved treatments, but alprostadil is being developed in a cream for treatment. Also, a transdermal testosterone gel has been tested clinically in women with surgically menopausal FSAD, as well as for those with low libido.

Menopause occurs when follicles disappear from a woman's ovaries, thereby eliminating the most productive estrogen source. Estrogen replacement therapy is utilized to treat the effects of menopause and includes the use of estrogen, progesterone, estradiol, progestin, and various synthetic estrogen derivatives (selective estrogen receptor modulators), such as raloxifene.

Finally, breast disorders such as benign growths and premenopausal pain can be treated with tamoxifen and afimoxifene, an antiestrogen

(xv) Nausea/Vomiting

A large number of anti-emetics are known and can be used to treat such ailements as motion/air/sea sickness. These include 5-HT₃ receptor antagonists (Dolasetron, Granisetron, Ondansetron, Tropisetron and Palonosetron), dopamine antagonists (Domperidone, Droperidol, Haloperidol, Chlorpromazine, Promethazine, Prochlorperazine, Metoclopramide, Alizapride), antihistamines (H₁ histamine receptor antagonists; Cyclizine, Diphenhydramine, Dimenhydrinate, Meclizine, Promethazine-Pentazine, Phenergan, Promacot, Hydroxyzine), cannabinoids such as Dronabinol (Marinol), Nabilone (Cesamet), and Sativex, benzodiazepines (Midazolam, Lorazepam) anticholinergics (Hyoscine, also known as Scopolamine), steroids (Dexamethasone) Trimethobenzamide, Ginger, Emetrol, Propofol, Peppermint, Muscimol

(xvi) Smoking

Perhaps the most widely applied of all transdermal treatments is the use of nicotine replacement patches to assist in the cessation of smoking. Bupropion and clonidine also have been indicated for this use.

(xvii) Contraception

Female contraceptives can be delivered across tissue surfaces. These include levonorgestrel and norelgestromin/ethinyl estradiol. Androgens and progestagens have recently been shown to suppress spermatogenesis and thus may provide the opportunity for male contaceptive devices.

(xviii) Vitamins

Vitamins, such as Vitamin A, Vitamin B complex, Vitamin C, Vitamin D, Vitamin E, and Vitamin K, all provide benefit to subjects in general, and may also help prevent or treat certain disease states, and thus are candidates for topical/transdermal delivery.

B. Cosmetic/Cosmeceutic Agents

Cosmetics are substances used to enhance or protect the appearance or odor of the human body. Cosmetics include skin-care creams, lotions, powders, perfumes, lipsticks, fingernail and toenail polishes, eye and facial makeup, permanent waves, hair colors, hair sprays and gels, deodorants, baby products, bath oils, bubble baths, bath salts, butters and many other types of products. Their use is widespread, especially among women in Western countries. A subset of cosmetics is called “make-up,” which refers primarily to colored products intended to alter the user's appearance. Cosmeceuticals are cosmetic products that are claimed to have drug-like benefits. Examples of products typically labeled as cosmeceuticals include anti-aging creams and moisturizers. Cosmeceuticals often are said to contain purported active ingredients such as vitamins, phytochemicals, enzymes, antioxidants, and essential oils.

The CTFA International Cosmetic Ingredient Dictionary and Handbook (2004) describes a wide variety of non-limiting cosmetic ingredients that can be used in the context of the present invention. Examples of these ingredient classes include fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraaminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided in the following subsections.

Colorants

In certain non-limiting aspects, the guar gum containing compounds can be used to efficiently disperse colorants throughout a composition and/or a phase (e.g., water, oil, silicone phase) of the composition. Non-limiting examples of colorants that can be used in the context of the present invention include those known to a person of ordinary skill in the art (see, e.g., CTFA International Cosmetic Ingredient Dictionary and Handbook, 2004). For instance natural and synthetic pigments and lakes can be used. Examples of groups of pigments include carbon, cadmium, iron oxide, Prussian blue, chromium, cobalt, copper, titanium, ultramarine, zinc, clay earth, and organic pigments. Specific non-limiting examples of colorants include Aluminum Powder, Blue 1 Lake, Bronze Powder, Chromium Oxide Greens, Copper Powder, Ext. Yellow 7 Lake, Green 3 Lake, Orange 4 Lake, Orange 5 Lake, Orange 10 Lake, Pigment Blue 15, Pigment Blue 15:2, Pigment green 7, Pigment Orange 5, Pigment Red 4, Pigment Red 5, Pigment Red 48, Pigment Red 53, Pigment Red 53:1, Pigment Red 57, Pigment Red 57:1, Pigment Red 63:1, Pigment Red 64:1, Pigment Red 68, Pigment Red 83, Pigment Red 88, Pigment Red 90:1 Aluminum Lake, Pigment Red 112, Pigment Red 172 Aluminum Lake, Pigment Red 173 Aluminum Lake, Pigment Red 190, Pigment Violet 19, Pigment Yellow 1, Pigment Yellow 3, Pigment Yellow 12, Pigment Yellow 13, Pigment Yellow 73, Red 4 Lake, Red 6 Lake, Red 7 Lake, Red 21 Lake, Red 22 Lake, Red 27 Lake, Red 28 Lake, Red 30 Lake, Red 31 Lake, Red 33 Lake, Red 34 Lake, Red 36 Lake, Red 40 Lake, Sunset Yellow Aluminum Lake, Yellow 5 Lake, Yellow 6 Lake, Yellow 7 Lake, Yellow 10 Lake, and Zinc Oxide.

(ii) Preservatives

Non-limiting examples of preservatives that can be used in the context of the present invention include quaternary ammonium preservatives (e.g., polyquaternium-1), parabens (e.g., methylparabens and propylparabens), phenoxyethanol, benzyl alcohol, chlorobutanol, phenol, sorbic acid, thimerosal or combinations thereof.

(iii) Moisturizers

Non-limiting examples of moisturizing agents that can be used with the compositions of the present invention can be found in the International Cosmetic Ingredient Dictionary, 10^{th} ed. (2004). Examples include amino acids, chondroitin sulfate, diglycerin, erythritol, fructose, glucose, 1,2,6-hexanetriol, honey, hyaluronic acid, hydrogenated honey, hydrogenated starch hydrolysate, inositol, lactitol, maltitol, maltose, mannitol, natural moisturizing factor, salts of pyrollidone carboxylic acid, potassium PCA, sodium glucuronate, sodium PCA, sorbitol, sucrose, trehalose, urea, xylitol, glycerin, and petrolatum.

(iv) Emollients

Non-limiting examples of emollients include, but are not limited to, vegetable oils, mineral oils, silicone oils, synthetic and natural waxes, petrolatum, lanolin, aluminum magnesium hydroxide stearate (which can also function as a water repellent), and fatty acid esters. Non-limiting examples of vegetable oils include safflower oil, corn oil, sunflower seed oil, olive oil, or joboba esters.

(v) Antioxidants

Non-limiting examples of antioxidants include, but are not limited to, acetyl cysteine, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

(vi) Thickening Agents

Thickening agents, including thickener or gelling agents, include substances which that can increase the viscosity of a composition. Thickeners include those that can increase the viscosity of a composition without substantially modifying the efficacy of the ingredients within the composition. Thickeners can also increase the stability of the compositions of the present invention. Non-limiting examples of additional thickeners that are known to those of ordinary skill in the art can be used in the context of the present invention (e.g., U.S. Pat. Nos. 5,087,445; 4,509,949; 2,798,053; International Cosmetic Ingredient Dictionary and Handbook, 10^{th} Ed., 2004). Examples include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

(vii) Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. The silicon containing compound can be a silicone oil such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e., normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in International Cosmetic Ingredient Dictionary, 10^{th} ed., 2004 as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div., Stauffer Chemical Co., Adrian, Mich.

### V. FORMULATIONS, DELIVERY VEHICLES AND ARTICLES OF MANUFACTURE

A. Formulations

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, media, coatings, antibacterial and antifungal agents, and the like. The use of such reagents in pharmaceuticals is well known in the art. Supplementary active ingredients also can be incorporated into the compositions.

Upon formulation, solutions will be administered in a manner compatible with the dosage formulation and in such amount as is therapeutically effective. The formulation of choice can be accomplished using a variety of excipients including, for example, pharmaceutical grades of mannitol, lactose, starch, magnesium stearate, sodium saccharin cellulose, magnesium carbonate, and the like.

Typically, the compounds of the instant invention will contain from less than 1% to about 95% of the active ingredient, preferably about 10% to about 50%. The frequency of administration will be determined by the care given based on patient responsiveness. Other effective dosages can be readily determined by one of ordinary skill in the art through routine trials establishing dose response curves.

B. Devices and Delivery Sites

In accordance with the present invention, there will be provided various devices and preparations that will assist in topical, transdermal and percutaneous delivery of the avicin/agent compositions described herein. Although devices and formulations that impart their own effects on transport maybe utilized, it is not necessary that the devices or preparations used herein provide any more than a structural role to contain the avicin/agent compositions and to provide a means of bringing such compositions into contact with the appropriate tissue.

In one topical embodiment, the present invention can utilize a patch. A transdermal or “skin” patch is a medicated adhesive patch that is placed on the skin to deliver a time released dose of medication through the skin and into the bloodstream. A wide variety of pharmaceuticals can be delivered by transdermal patches. The first commercially available prescription patch was approved by the U.S. Food and Drug Administration in December 1979, which administered scopolamine for motion sickness.

The highest selling transdermal patch in the United States is the nicotine patch which releases nicotine to help with cessation of tobacco smoking. Other skin patches administer estrogen for menopause, which also seems to prevent osteoporosis after menopause. Nitroglycerin patches for angina and lidocaine patches to relieve the peripheral pain of shingles (herpes zoster) are also available. Recent developments expanded the use of patches to the delivery of hormonal contraceptives, antidepressants, pain killers and stimulants for Attention Deficit Hyperactivity Disorder/ADHD.

The main components to a transdermal patch are (a) a liner to protect the patch during storage (removed prior to use); (b) the active agent; (c) an adhesive that serves to adhere the components of the patch together along with adhering the patch to the skin; (d) a membrane to control the release of the drug from the reservoir and multi-layer patches; and (e) a backing that protects the patch from the outer environment.

There are four main types of transdermal patches. Single-layer Drug-in-Adhesive patches have an adhesive layer that also contains the agent. In this type of patch the adhesive layer not only serves to adhere the various layers together, along with the entire system to the skin, but is also responsible for the releasing of the drug. The adhesive layer is surrounded by a temporary liner and a backing. Multi-layer Drug-in-Adhesive patches are similar to the single-layer system in that both adhesive layers are also responsible for the releasing of the drug. The multi-layer system is different however that it adds another layer of drug-in-adhesive, usually separated by a membrane (but not in all cases). This patch also has a temporary liner-layer and a permanent backing. Reservoir patches are unlike the Single-layer and Multi-layer Drug-in-Adhesive systems in that the reservoir transdermal system has a separate drug layer. The drug layer is a liquid compartment containing a drug solution or suspension separated by the adhesive layer. This patch is also backed by the backing layer. In this type of system the rate of release is zero order. Matrix patches have a drug layer of a semisolid matrix containing a drug solution or suspension. The adhesive layer in this patch surrounds the drug layer partially overlaying it.

Various alternate sites of administration will also find use with the subject invention. For instance, the compositions of the invention may be formulated, in addition to the formulations discussed above, in suppositories, douches, aerosol and intranasa; compositions. Intranasal formulations may be prepared which include vehicles that neither cause irritation to the nasal mucosa nor significantly disturb ciliary function. Diluents such as water, aqueous saline or other known substances can be employed with the subject invention. The nasal formulations also may contain preservatives such as, but not limited to, chlorobutanol and benzalkonium chloride.

In certain embodiments, a cancer may treated after surgical excision to eliminate microscopic residual disease. Both at the time of surgery, and thereafter (periodically or continuously), the therapeutic compositions of the present invention can be administered to the body cavity. This is, in essence, a topical treatment of the surface of the cavity. The volume of the composition should be sufficient to ensure that the entire surface of the cavity is contacted by the expression construct. By analogy, a post-operative field may be treated to prevent or inhibit infection.

In one embodiment, administration simply will entail injection of the therapeutic composition into the cavity formed by the surgery/tumor excision. In another embodiment, mechanical application via a sponge, swab or other device may be desired. Either of these approaches can be used subsequent to the tumor removal as well as during the initial surgery. In still another embodiment, a catheter is inserted into the cavity prior to closure of the surgical entry site. The cavity may then be continuously perfused for a desired period of time.

In another form of this treatment, the topical application of the therapeutic composition is targeted at a natural body cavity such as the mouth, pharynx, esophagus, larynx, trachea, pleural cavity, peritoneal cavity, or hollow organ cavities including the bladder, colon or other visceral organs. Again, a variety of methods may be employed to affect the topical application into these visceral organs or cavity surfaces. For example, the oral cavity in the pharynx may be affected by simply oral swishing and gargling with solutions.

Many inflammatory diseases will also be amenable to the topical application of the therapeutic composition to a natural body cavity such as the mouth, pharynx, esophagus, larynx, trachea, pleural cavity, peritoneal cavity, or hollow organ cavities including the bladder, colon or other visceral organs. For example, topical application to the intestinal epithelium may be used in the treatment of inflammatory bowel disorders, such as Crohn's disease and ulcerative colitis. As another example, topical application to the bladder could be useful for the treatment of diseases, such as interstitial cystitis.

C. Vehicles

In another embodiment, the present invention will utilize a fluid or semi-fluid vehicle. Non-limiting examples of suitable vehicles include emulsions (e.g., water-in-oil, water-in-oil-in-water, oil-in-water, oil-in-water-in-oil, oil-in-water-in-silicone, water-in-silicone, silicone-in-water emulsions), creams, lotions, solutions (both aqueous and hydro-alcoholic), anhydrous bases (such as lipsticks and powders), gels, powdered and liquid aerosols, ointments, and other combination of the forgoing as would be known to one of ordinary skill in the art (see, e.g., Remington's, 1990 and International Cosmetic Ingredient Dictionary and Handbook, 10^{th} ed., 2004). Variations and other appropriate vehicles will be apparent to the skilled artisan and are appropriate for use in the present invention. In certain aspects, it is important that the concentrations and combinations of the compounds, ingredients, and agents be selected in such a way that the combinations are chemically compatible and do not form complexes which precipitate from the finished product.

The present invention can also utilize many cosmetic articles of manufacture, but not limited to, sunscreen products, sunless skin tanning products, hair products, finger nail products, moisturizing creams, skin benefit creams and lotions, softeners, day lotions, gels, powders, aerosols, ointments, foundations, night creams, lipsticks, cleansers, toners, masks, or other known cosmetic products or applications. Additionally, the cosmetic products can be formulated as leave-on or rinse-off products. In certain aspects, the compositions of the present invention are stand-alone products.

### VI. THERAPEUTIC INDICATIONS

A. Cancer

In one aspect, the present invention will find use in the treatment of cancers found on the exposed surfaces and membranes of a living organism. In particular, the treatment of skin cancers, such as melanoma, is contemplated. However, as discussed above, a variety of cancers may benefit from treatment with compositions of the present invention, including breast, brain, stomach, esophageal, liver, pancreas, colon, rectal, prostate, uterine, cervical and heac & neck cancers. The cancers may be primary or metastatic, recurrent, or multi-drug resistant.

Skin cancer is a malignant growth on the skin which can have many causes. Skin cancer generally develops in the epidermis (the outermost layer of skin), so a tumor is usually clearly visible. This makes most skin cancers detectable in the early stages. There are three common types of skin cancer, each of which is named after the type of skin cell from which it arises. Cancers caused by UV exposure may be prevented by avoiding exposure to sunlight or other UV sources, wearing sun-protective clothes, and using a broad-spectrum sun screen. Skin cancers are the fastest growing type of cancer in the United States. Skin cancer represents the most commonly diagnosed malignancy, surpassing lung, breast, colorectal and prostate cancer, with more than 1 million Americans being diagnosed with skin cancer in 2007.

The most common types of skin cancer are basal cell carcinoma (BCC) and squamous cell carcinoma (SCC) which may be locally disfiguring but are unlikely to metastasize (spread to other parts of the body). The most dangerous type of skin cancer is malignant melanoma. This form of skin cancer can be fatal if not treated early but comprises only a small proportion of all skin cancers. More rare types of skin cancer include Dermatofibrosarcoma protuberans, Merkel cell carcinoma and Kaposi's sarcoma.

In the context of the treatment of solid tumors, it is contemplated that effective amounts of the compositions of the invention will be those that generally result in at least about 10% of the cells within a tumor exhibiting cell death, apoptosis, or tissue necrosis. In various embodiments, at least about 20%, about 30%, about 40%, or about 50% of the cells at a particular tumor site will be killed, with 100% of the tumor cells at a tumor site being the ideal objective, as well as at least about 20%, about 30%, about 40%, or about 50% of the tumor will become necrotic, with 100% necrosis as the ideal objective.

B. Skin/Muscosal Infections

A variety infections take place at the exterior surface of a tissue, such as the skin, mouth, vagina, or urethra, or in a post-surgical cavity or other open wound. Such infections may be caused by viruses, fungi or even parasites, but are most commonly caused by bacteria.

Impetigo, a vesicular infection of the skin, is usually caused by Staphylococcus aureus, but sometimes Streptococcus pyogenes (group A streptococci) is also present. Occasionally, S. pyogenes is the sole responsible organism. For treatment of sparse lesions, topical mupirocin ointment is as effective as oral antimicrobials. Systemic antibiotics active against both S. aureus and S. pyogenes, such as cephalexin or dicloxacillin, are an alternative to topical treatment and are preferred to topical therapy for extensive lesions. Ecthyma is a deeper infection than impetigo. Treatment should be with an oral antistaphylococcal agent, such as dicloxacillin or cephalexin.

Cellulitis and erysipelas are acute, spreading infections of the skin caused by streptococci of groups A, B, C, and G. Erysipelas involves the superficial dermis, especially the dermal lymphatics, and cellulitis affects the deeper dermis and subcutaneous fat. Infection commonly occurs on skin that has been permanently damaged by burns, trauma, radiotherapy, or surgery. For example, cellulitis may occur at the site of a saphenous vein removal for cardiac or vascular surgery months to years after the procedure. Obesity is also a predisposing condition. Treatment consists of elevation of the affected area to help reduce edema and administration of systemic antibiotic therapy. For patients who do not have serious systemic illness, oral treatment is satisfactory. Penicillin is the drug of choice for streptococcal infections, but many clinicians prescribe an antistaphylococcal agent, such as a first-generation cephalosporin or a penicillinase-resistant penicillin, because of concerns about S. aureus.

Furunculosis results in a deep-seated inflammatory nodule with a pustular center that develops around a hair follicle. With involvement of several adjacent follicles, a mass called a carbuncle may form, with pus discharging from multiple follicular orifices. About 20% to 40% of the population carry S. aureus in the anterior nares. From this site or occasionally from the perineum or axilla, organisms can spread and enter the skin, presumably through minor, usually inapparent, trauma. Clindamycin, when given as a single daily dose of 150 mg for 3 months, is very effective in preventing subsequent episodes. A less effective alternative is mupirocin ointment.

Cutaneous abscesses are collections of pus within the dermis and deeper skin tissues and probably occur as a result of trauma. S. aureus, usually in pure culture, causes about 25% of cutaneous abscesses, but anaerobes can also be involved (fecal bacteria, including streptococci, anaerobic gram-positive cocci, and anaerobic gram-negative bacilli, such as Bacteroides fragilis). Treatment is incision and drainage of the area. Gram stain and culture of the pus are ordinarily unnecessary, as are topical antimicrobials. Systemic antibiotics are reserved for patients with extensive surrounding cellulitis, neutropenia, cutaneous gangrene, or systemic manifestations of infection, such as high fever.

Porphyrin-producing coryneform bacteria, which are gram-positive bacilli that constitute part of the normal cutaneous flora, cause a superficial, usually asymptomatic, skin disorder called erythrasma. Corynebacterium minutissimum, has often been cited as the sole cause of this infection, but its precise role, if any, remains unclear. Because they possess some activity against gram-positive bacteria, topical azoles, such as miconazole and clotrimazole, are effective in the treatment of this infection. Topical erythromycin or clindamycin is also effective, as is oral erythromycin.

C. minutissimum and a gram-positive coccus, Micrococcus sedentarius, either alone or together, cause a disorder called pitted keratolysis, small pitted erosions about 1 to 7 mm in diameter on the soles of the feet. As in erythrasma, topical azoles, such as clotrimazole and miconazole, are effective, as are topical erythromycin and clindamycin.

Trichomycosis axillaris is characterized by colored concretions of axillary hair that result from infection of the hair shafts by large colonies of various species of Corynebacterium. Shaving the hair is effective treatment; other options include topical erythromycin or clindamycin.

Necrotizing fasciitis, a necrotizing infection of the subcutaneous tissue, can be caused by streptococci; more often, however, the responsible organisms are a combination of aerobic bacteria (gram-negative enteric organisms (e.g., Escherichia coli), gram-positive cocci, and anaerobes, including B. fragilis). Necrotizing fasciitis usually occurs after a penetrating wound to the extremities. Gram stain and culture dictate antibiotic choice, but gentamicin in combination with clindamycin is a first choice. Also, incision and drainage of the affected area is recommended.

Folliculitis is an inflammation at the opening of the hair follicle that causing erythematous papules and pustules surrounding individual hairs. Among bacteria, S. aureus is often suspected but rarely found. Another cause is M furfur, a yeast that is a normal resident on the skin. Pseudomonas aeruginosa may also be responsible, as a consequence of inadequate disinfection of hot tubs, swimming pools, or whirlpools.

Spores of Bacillus anthracis sent through the mail in the fall of 2001 as an act of bioterrorism caused cases of inhalational and cutaneous anthrax in several states. Otherwise, anthrax has been very rare in the United States over the past few decades. The cutaneous form develops when spores enter the skin through abrasions and then transform into bacilli, which produce toxins that cause local tissue edema and necrosis. Treatment is penicillin V (500 mg q.i.d. orally) or amoxicillin (500 mg t.i.d. orally) for mild cases and, for more severe disease, penicillin G (6 to 8 million units I.V. daily). Oral ciprofloxacin (500 mg b.i.d.) or doxycycline (100 mg b.i.d) is available as well.

C. Cardiovascular Disease

A variety of drugs are currently delivered across tissue surfaces to patients with cardiovascular disease, including angina pectoris, high blood pressure, cardiac hypertrophy, and heart failure. For example, sublingual nitroglycerin has been used for the prevention of angina pectoris in coronary artery heart disease. Transdermal administration would overcome some of these drawbacks of systemic administration of nitroglycerin. Various preparations of transdermal nitroglycerin are available and some are in development.

Hypertension, known as the “silent killer” since it shows few, if any, symptoms, is a major risk factor for other cardiovascular diseases such as stroke, heart attacks and congestive heart failure. It is possible to effect reasonable management of chronic hypertension with the use of chronic transdermal clonidine therapy.

Beta-adrenoceptor blocking drugs (β-blockers) are one of the most frequently used class of cardiovascular drugs that are mainly used in conventional dosage forms, which have limitations such as hepatic first-pass metabolism, high incidence of adverse effects due to variable absorption profiles, higher frequency of administration and poor patient compliance. So far no β-blockers have been marketed as transdermal delivery systems. Penetration-enhancing strategies, such as iontophoresis, electroporation, microneedles and sonophoresis, have been discussed, but a more direct approach such as the inclusion of avacins in the delivery vehicle, would be advantageous.

Heparin and low-molecular weight heparin are the most commonly used anticoagulants and are administered by intravenous or subcutaneous injections. However, injections of heparin have the potential risk of bleeding complications and the requirement of close monitoring in some cases. Transdermal drug delivery offers an attractive alternative to injections due to minimization of pain, sustained release of drugs, control of the rate of administration, and termination on demand. However, the dose of transdermally delivered heparin is limited by low skin permeability. Thus, agents that promote heparin transfer could render this form of delivery more viable.

Levosimendan (Simdax®) is one of the first agents of a new class of drugs known as calcium sensitizers. It is approved for the treatment of heart failure in some European countries and is in phase III clinical trials in the U.S. The mechanism of action is an increased cardiac contractility by sensitizing cardiac myofibrils to calcium. Transdermal delivery of levosimendan can be significantly increased by formulation modification (Valjakka-Koskela et al., 2000). Based on kinetic calculations, therapeutic plasma concentrations may be achievable transdermally.

D. Contraception

The present invention may be used advantageously to deliver agents that act as contraceptives. While these uses have heretofore be limited to female contraception, male contraceptives may prove useful as well.

Female

Ortho Evra™ (Johnson & Johnson) is the first transdermal contraceptive approved by the FDA in 2001 and marketed in 2002. Containing norelgestromin/ethinyl estradiol, it is a once-a-week birth control option that is purported to be as effective (99%) as oral contraceptives. In the U.S., more than four million women have used the patch since it went on sale in 2002. It was also introduced in the UK. Results from a survey released on in 2003 and conducted by Ortho-McNeil Pharmaceutical, maker of the patch, indicated that 9 out of 10 patch users prefer Ortho Evra™ to their former birth control method, and 95% of women were satisfied with Ortho Evra™ as a discreet form of birth control. The patients like the once-a-week schedule; it is convenient and easy to remember. The only known major rival Ortho Evra™ is a patch being developed by Schering AG, but the company has not yet filed for marketing authorization. Levonorgestrel/ethinyl estradiol-contraceptive Patch (Agile Therapeutics) is in phase II clinical trials.

(ii) Male

Recent studies demonstrate that combinations of androgens and progestagens are highly effective in the suppression of spermatogenesis in normal volunteers. One study compared Norplant II and testosterone transdermal patch to T patch alone on the suppression of spermatogenesis in normal men to test whether progestagen and androgen delivery systems designed to produce steady serum levels will be as effective as other androgen plus progestagen combinations (Gonzalo et al., 2002). Although more efficacious than transdermal patch alone, Norplant II or oral levonorgestrel plus transdermal patch was not as effective in suppressing spermatogenesis to severe oligo- or azoospermia. The study concluded that Norplant II implants plus testosterone enanthate 100 mg/wk were very efficient in suppressing spermatogenesis to a level acceptable for contraceptive efficacy. This study demonstrates that the dose or route of administration of androgens is critical for sperm suppression in combined androgen-progestagen regimens for hormonal male contraception.

E. Pain

Treatment of pain, particularly chronic pain, constitutes an immense undertaking for the medical field. The present invention may advantageously be used to deliver drugs to treat pain given that transdermal devices may be used to deliver a steady amount of drug to a patient over a set period of time, and can also be used to target particular areas of the body. Particular aspects of pain relief include pain associated with cancer and cancer therapy, post-surgical pain, post-trauma pain, pain associated with minor surgical procedures, other neuropathic pain (herpes zoster infection, HIV-AIDS, toxins, alcoholism) and arthritis (osteo- and rheumatoid).

F. Cosmeceutic Applications

In still additional embodiments, the present invention can be used to improve the delivery of cosmetic or cosmeceutial agents. In particular, the present invention will find utility in delivering moisturizing agents, anti-aging compounds (e.g., anti-oxidants), and UV-protectors to the skin.

### VII. ASSAYS AND METHODS FOR SCREENING FOR ENHANCEMENT OF AGENT DELIVERY

In another aspect, the present invention provides for assays to assess the ability of an avicin or avicin composition to enhance the transdermal, topical or percutaneous delivery of agents. These include assays of biological activities as well as assays of chemical properties. Assays may be conducted in vivo or in vitro and may assess transport directly, by examining the location or relative amount of an agent in a target site, or by looking at a biological effect of the agent in the target site.

An exemplary method used by the inventors for carrying out such assays include the following.

Transdermal Transport Assays

Full thickness porcine skin was harvested from the back and sides of a female pig, and were procured from Lampire Inc. Sections of skin were thawed, then one and a half inch by one and a half inch samples were cut to be mounted on side by side diffusion cells. Each diffusion cell set up had a “donor compartment” which faced the stratum corneum side of the skin and was loaded with various drug formulations with a radiolabled species. The “receiver compartment” which faces the dermis side of the skin was loaded with 2 ml of PBS, and sampled for the transport of the radiolabeled species over a time course. At the beginning of each experiment 20 ul was taken from each donor compartment to verify starting drug concentrations. At each sampling time during the time course of the experiment, the full 2 ml volume of the receiver compartment was removed and stored for analysis, and then a fresh 2 ml of PBS was replaced. At the end of the experiment the donor compartment was sampled to determine the remaining amount of the species of interest.

F094 a crude mixture of Avicins, Avicin D and Avicin G were procured from the Clayton foundation and shipped in lyophilized form. Upon receipt, concentrated stock solutions were made in DMSO. All experiments were run in triplicate (N=3) and each experiment was repeated 2-3 times to ensure all trends remained consistent.

Analysis of radioactive species transport by scintillation counting

Each 2 ml receiver compartment sample at each time point was transferred into a scintillation vial and 2 ml of scintillation fluid was added. Each sample was run through a Beckman LS6500 beta counter, and raw scintillation counts of radioactivity were printed out. Known concentrations of tritiated (³H) water, ³H-estradiol, ¹²⁵I-Avicin were used to create standard concentration curves to correlate radioactive measurements to mass. Data analysis was done in Excel, and data was reported as averages+/−standard deviation to represent all replicates. Flux and permeability values were calculated based on values within steady state transport regions.

It will naturally be understood that combinations of agents intended for use together should be tested and optimized together. The compounds of the invention can be straightforwardly analyzed in combination with one or more therapeutic drugs or diagnostic agents with an avicin compound or composition. Analysis of the combined effects on transport would be determined and assessed according to the guidelines set forth above.

### VIII. KITS

Kits of the present invention may have a single container that contains the avicin compounds or compositions, with or without any additional components, or they may have distinct container means for each desired agent. When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent also may be provided in another container means. The container means of the kit will generally include at least one vial, test tube, flask, bottle, syringe or other container means, into which the avicin composition may be placed and suitably aliquoted. Where additional components (e.g., therapeutic or diagnostic agents) are included, the kit will also generally contain a second vial or other container into which these are placed. The kits also may comprise a second/third container means for containing a sterile, pharmaceutically acceptable buffer or other diluent.

The kits also may contain a means by which to administer the compositions to an animal or patient, e.g., one or more devices or apparati. The kits of the present invention will also typically include a means for containing the vials, or such like, and other component, in close confinement for commercial sale, such as, e.g., cardboard containers or injection or blow-molded plastic containers into which the desired vials and other apparatus are placed and retained.

### IX. EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

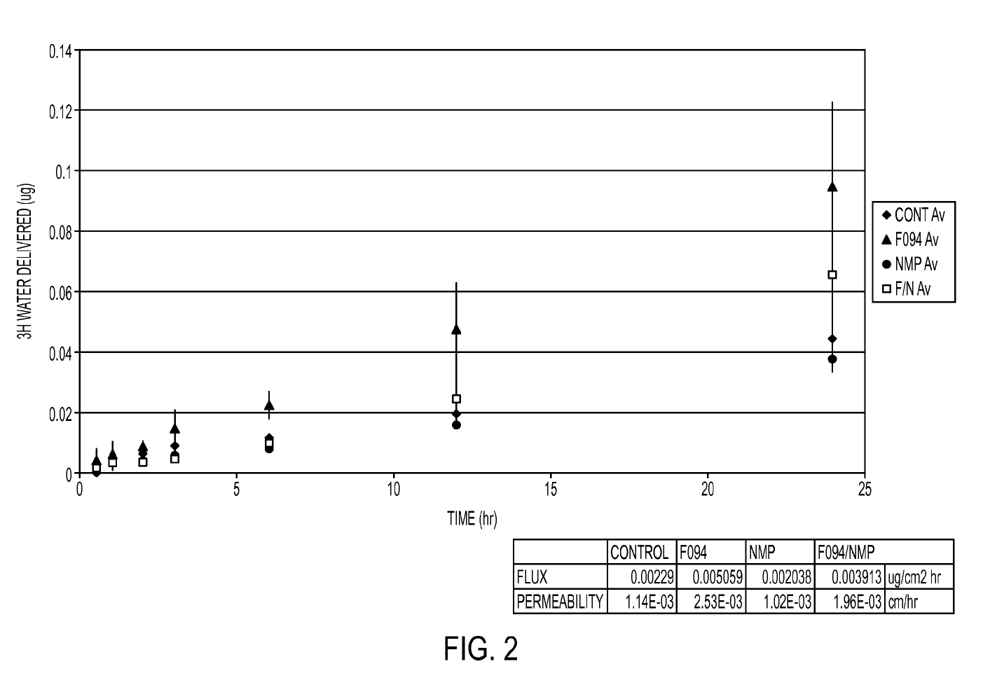

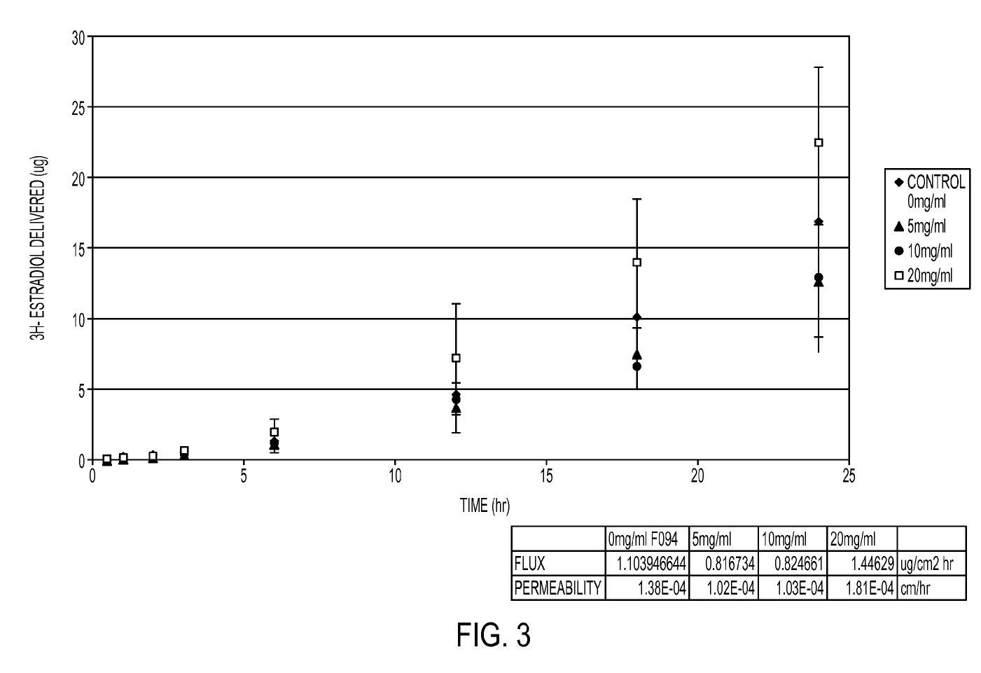

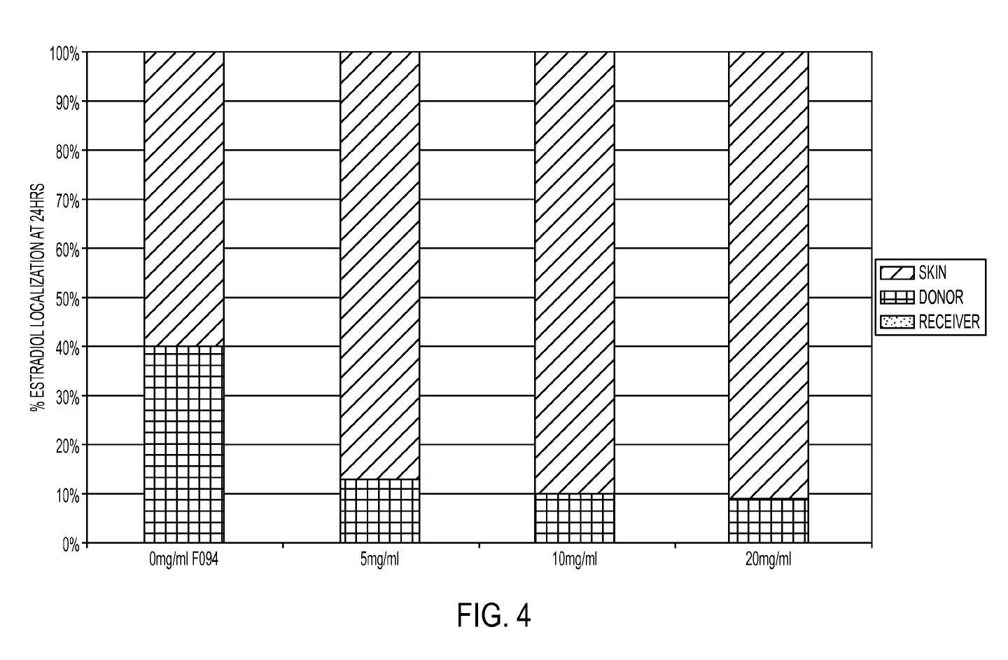

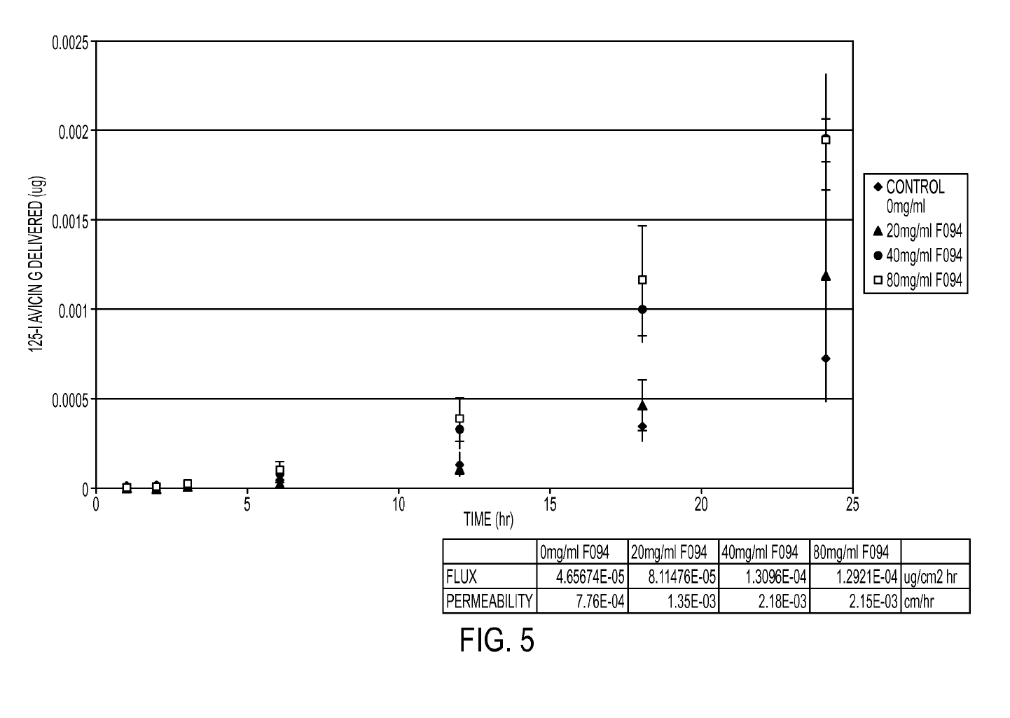

The inventors have ample evidence that F094, a crude mixture of Triterpene saponins from Acacia Victoriae that contains ˜15% Avicin D and ˜5% Avicin G (Jayatilake 2003), is able to significantly increase both water transport and estradiol transport through full thickness pig skin in vitro (FIGS. 2-3). In addition, estradiol percutaneous absorption is increased when co-administered with Avicins (FIG. 4). The inventors have also conducted experiments showing that radiolabeled Avicin G, which is molecular weight, traverses full thickness skin by itself, without the presence of an enhancer in the formulation (FIG. 5). The amount of Avicin G delivered, has be previously shown to be efficacious in arresting tumorigenesis.

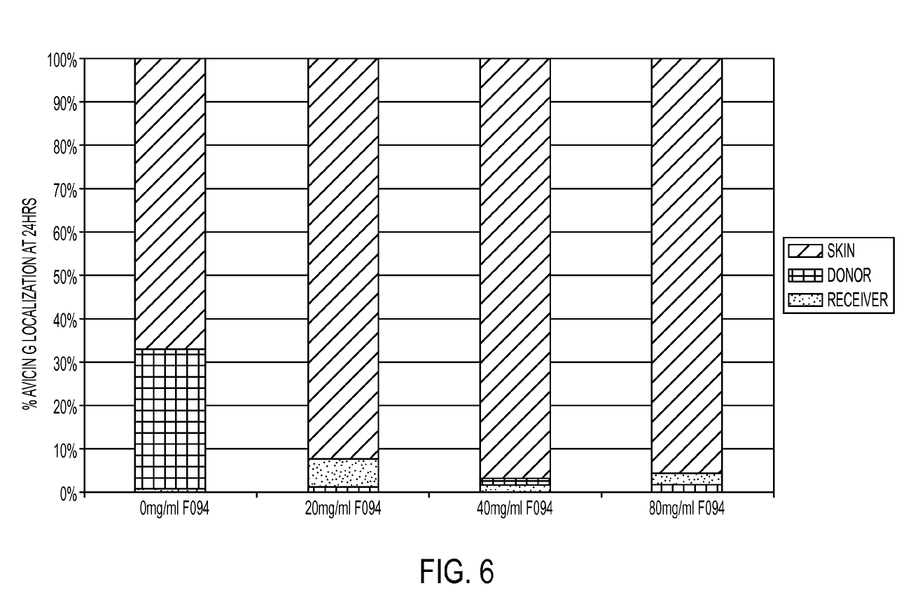

The inventors observed dramatic differences in how Avicin G and Avicin D affect water transport across full thickness hydrated pig skin. Avicin G, the form that is believed to possess greater anti-tumorigenic properties. in fact inhibits water transport in comparison to Avicin D (which also exhibits antitumor activity). This is a significant finding, that has not been described in the literature before as the only difference between Avicin D and Avicin G lies in a R-group that is H versus OH, respectively (FIG. 1). This could be leveraged in the development of topical formulations to minimize water loss (dehydration due to evaporation) of skin or increase hydration of skin, respectively. Such agents can find many uses in cosmeceutical formulations. The inventors also found that when Avicin G was administered with the crude extract, F094, percutaneous absorption of Avicin G was increased (FIG. 6).

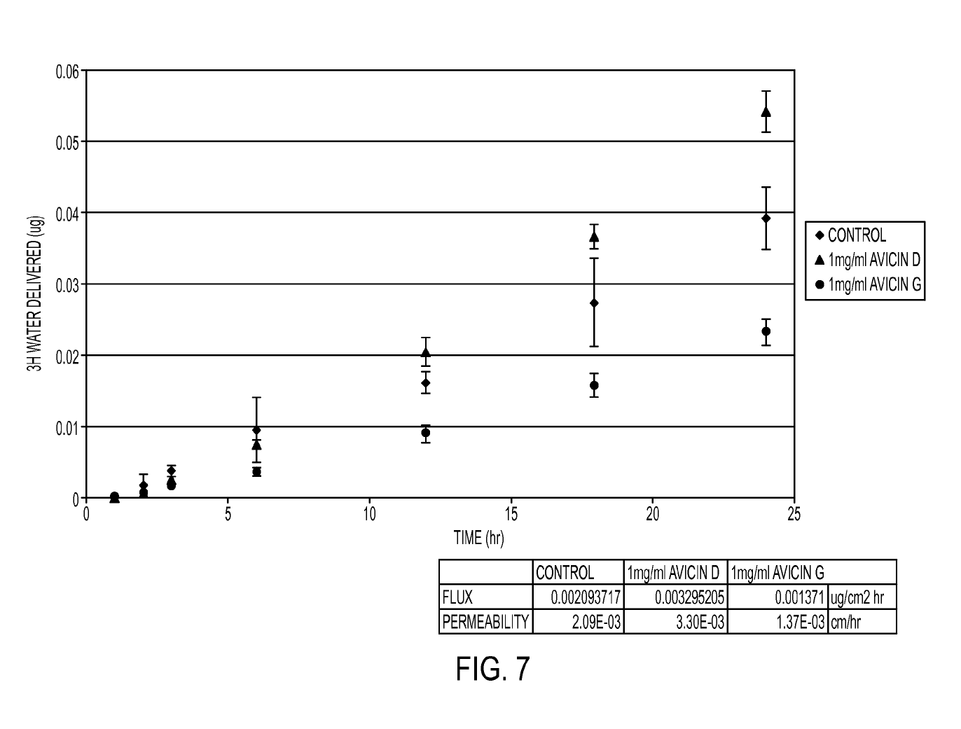

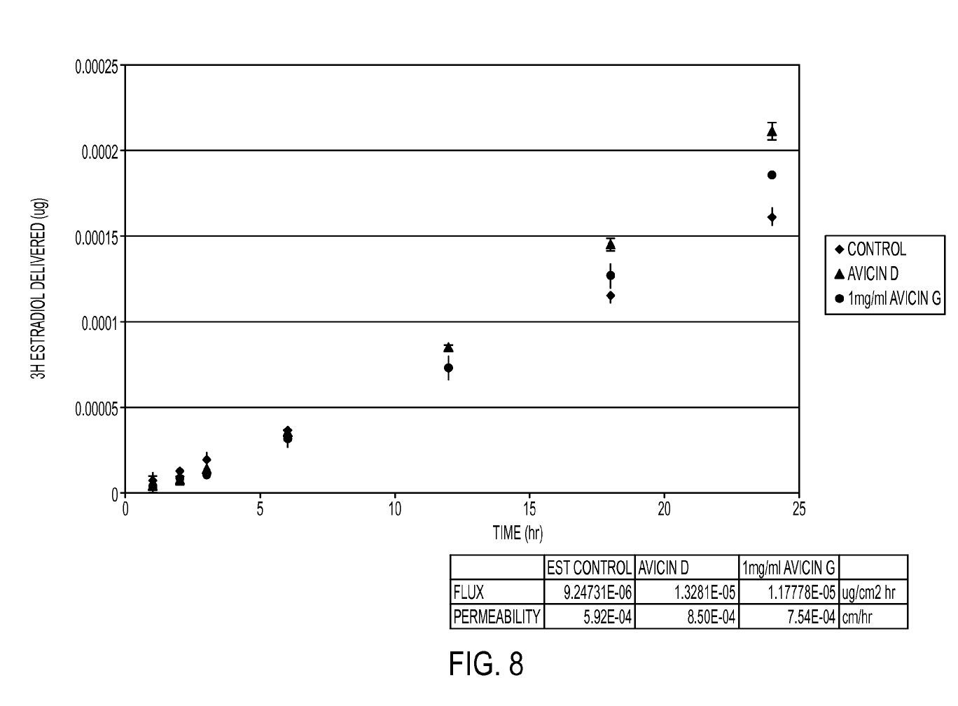

Recently, the inventors found that pure Avicin D is a potent enhancer of water and estradiol permeability through the skin, accounting for a portion of F094′s enhancing effects (FIGS. 7-8). Interestingly, Avicins and F094 are capable of enhancing both hydrophilic and hydrophobic molecules, which place it in a unique class of CPE's. Furthermore, CPE's tend to be small molecules never larger than 500 Daltons. Future effort will focus on evaluating the percutaneous adsorption characteristics of these molecules and mixtures in hairless mouse and rat models, towards one of its potential applications, treatment of skin cancers such as squamous cell carcinoma and melanomas.

### Example 2

Many anesthetics are currently applied topically on skin for local procedures. Water-soluble anesthetics (such as Liodcaine HCl and Prilocaine HCl) exhibit very limited percutaneous absorption and transdermal transport when delivered alone. Water soluble formulations of anesthetics are much preferred over lipophilic formulations, as the later cause skin irritation. Formulations with chemical penetration enhancers usually require that they constitute a large fraction of the mixture in order to be effective. Herein the inventors disclose the abilities of avicins in conjugation with triterpene saponins to enhance the permeability of water soluble drugs at donor concentrations as low as 0.1% w/v. Furthermore, the inventors disclose the enhancement of percutenous delivery of water soluble anesthetics, Lidocaine HCl, Bupivacaine HCl, and Prilocaine HCl, from a water based formulations containing 0.1 to 1% F094.

In order to deliver drugs approaching or larger than 300 Daltons (Lidocaine HCl, MW=271, Bupivacaine HCl, MW=343, and Prilocaine HCl, MW=257), mechanical methods of enhancement such as sonication and electroporation have been used (Prausnitz 1993, Boucaud 2002). In addition, chemicals known as chemical penetration enhancers (CPE) have been added to pharmaceutical formulations to enhance skin permeability. Many strategies have been employed to discover and characterize new enhancer molecules. The enhancers currently used are all lipophilic or lipid-like agents, so there remains a need to identify new classes of enhancers (Karande 2004). Despite the development of mechanical and chemical enhancers, the molecular weight cutoff of skin has not been substantially augmented (Finnin 1999, Purdon 2004).

F094 is a mixture of avicins and triterpene saponin glycosides that are extracted from a desert plant, Acacia victoriae. F094 is made up by 5% avicin D, 15% avicin G and 80% a mixture of triterpene saponins. Avicins have known anti-tumorigenic, antioxidant, anti-inflammatory, and antibiotic properties, that have been studied in depth (Haridas 2001, Haridas 2003, Haridas 2004, Gutterman 2005, Haridas 2007). Despite their high molecular weight ˜2,000 Daltons (Jayatilake 2003), the inventors have found that Avicins exhibit percutaneous absorption and transdermal transport. The inventors also have reported that avicin D and G differentially enhance water permeability.

In the studies, the inventors have found that formulations with F094 as low as 0.1% by weight, enhance the percutaneous absorption and transdermal delivery of selected water-soluble, aminoacyl anesthetics including Lidocaine HCl, Bupivacaine HCl, and Prilocaine HCl. This result is especially intriguing because a 0.1% aqueous solution containing enhancer has never before been shown to increase hydrophilic drug delivery. Most transdermal drug delivery systems require that the enhancer make up over 50% (by volume) of the formulation. Formulations with Avicins as enhancers have the potential to vastly expand the drugs able to be administered transdermally, both in the size of the drugs able to be delivered, but also drugs with previously unfavorable hydrophilic-lipophillic balance (HLB) and charge attributes. Aqueous formulations containing Avicins appear to significantly enhance hydrophilic drug transport, while maintaining attributes favorable to good skin interaction, minimizing irritation. Dermal sensitivity studies will be conducted, as well as animal studies showing efficacy of transdermal delivery.

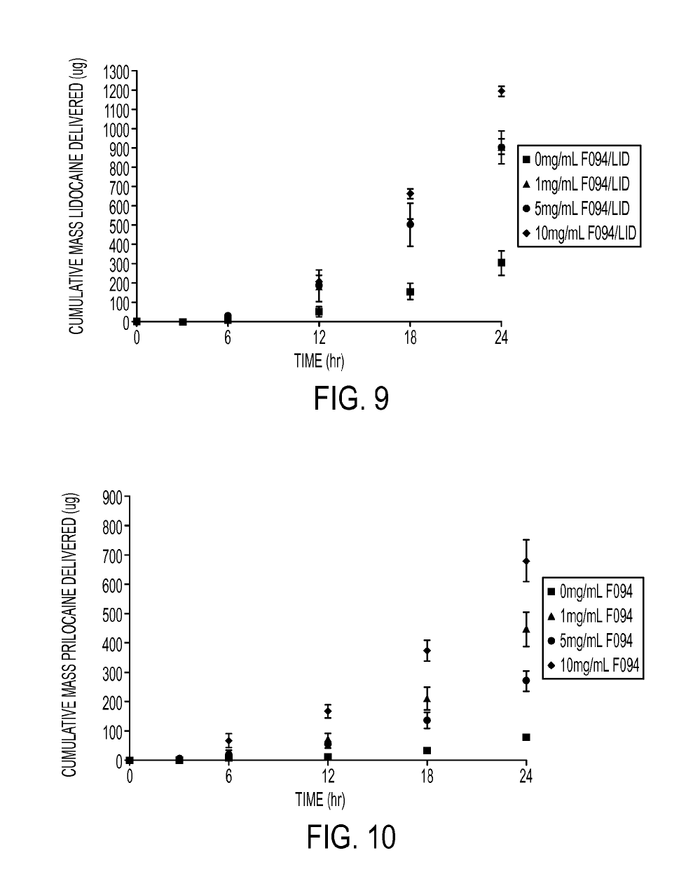

The inventors have provided ample evidence that F094, a crude mixture of Triterpene saponins from Acacia victoriae that contains −15% Avicin D and −5% Avicin G (Jayatilake 2003), is able to significantly increase Lidocaine HCl, Prilocaine HCl, and Bupivacaine HCl transport through full thickness pig skin in vitro (FIGS. 9-10, and Tables 1-4). In considering the demonstrated behavior of F094, the inventors have also confirmed, as summarized in Table 5, that although F094 enhances percutaneous absorption and transdermal delivery of lidocaine HCl, prilocaine HCl, and bupivacaine HCl, its inclusion in aqueous formulations does not impact the partioning of these anesthetics between octanol and water. This result suggests that the increased transdermal delivery of these anesthetics observed with increasing amounts of F094 is due to a change in the aqueous pathways within pigskin induced by F094. Thus, F094 may increase permeation by altering skin characteristics.

TABLE 1
LIDOCAINE HCL TRANSPORT IN FULL THICKNESS PIG SKIN

| | Permeability × | | |
|---|---|---|---|
| Donor compartment | 10⁻³ (cm/hr) | Lag time (hr) | % Enhancement |
| Control | 1.12 ± 0.31 | 10.12 ± 0.32 | NA |
| 1 mg/ml F094 | 2.62 ± 0.26 | 9.72 ± 0.25 | +134 ± 23% |
| 5 mg/ml F094 | 2.09 ± 0.85 | 10.61 ± 0.45 | +87 ± 75% |
| 10 mg/ml F094 | 3.08 ± 0.11 | 10.27 ± 0.12 | +175 ± 10% |

HPLC was used to quantify delivered mass, and to calculate permeabilities and lag times.
Values are mean +/− standard deviation.
Percent enhancement is a comparison measure within each experiment, a ratio of the permeability of the experimental sample and the permeability of the control.

TABLE 2
PRILOCAINE HCL TRANSPORT IN FULL THICKNESS PIG SKIN

| | Permeability × | | |
|---|---|---|---|
| Donor compartment | 10⁻³ (cm/hr) | Lag time (hr) | % Enhancement |
| Control | 0.46 ± 0.11 | 10.3 ± 1.1 | NA |
| 10 mg/ml F094 | 1.24 ± 0.23 | 10.7 ± 0.1 | +169 ± 50% |
| 20 mg/ml F094 | 1.36 ± 0.22 | 10.5 ± 0.1 | +196 ± 49% |
| 40 mg/ml F094 | 1.51 ± 0.31 | 9.9 ± 0.2 | +228 ± 67% |

HPLC was used to quantify delivered mass, and to calculate permeabilities and lag times.
Values are mean +/− standard deviation.
Percent enhancement is a comparison measure within each experiment, a ratio of the permeability of the experimental sample and the permeability of the control.

TABLE 3
BUPIVACAINE HCL TRANSPORT IN FULL
THICKNESS PIG SKIN

| | Permeability × | | % Enhancement |
|---|---|---|---|
| Donor compartment | 10⁻³ (cm/hr) | Lag time (hr) | Over control |
| Control | 0.71 ± 0.05 | 10.8 ± 1.1 | NA |
| 1 mg/ml F094 | 2.00 ± 0.23 | 9.9 ± 0.5 | +182 ± 32% |
| 5 mg/ml F094 | 1.97 ± 0.40 | 9.4 ± 0.1 | +177 ± 56% |
| 10 mg/ml F094 | 3.18 ± 0.62 | 9.5 ± 0.9 | +347 ± 87% |

HPLC was used to quantify delivered mass, and to calculate permeabilities and lag times.
Values are mean +/− standard deviation.
Percent enhancement is a comparison measure within each experiment, a ratio of the permeability of the experimental sample and the permeability of the control.

TABLE 4
COMPARISON OF WATER SOLUBLE ANESTHETIC
TRANSPORT IN FULL THICKNESS PIG SKIN

| | | Partition | Maximum |
|---|---|---|---|
| Donor | | coefficient | % Enhancement |
| compartment | Molecular weight | (octanol/water) | over control |
| Prilocaine HCl | 257 | 0.43 | 228% |
| Lidocaine HCl | 271 | 5.1 | 175% |
| Bupivacaine HCl | 325 | 1.7 | 347% |

HPLC was used to quantify delivered mass, and to calculate permeabilities and lag times.
Values are mean +/− standard deviation.
Percent enhancement is a comparison measure within each experiment, a ratio of the permeability of the experimental sample and the permeability of the control.

TABLE 5
PARTITION COEFFICIENT OF LIDOCAINE HCL
BETWEEN OCTANOL AND WATER

| Anesthetic | Amount of F094 | Partition Coefficient |
|---|---|---|
| Lidocaine HCl | 0 mg/ml F094 | 11.2 ± 0.6 |
| | 1 mg/ml F094 | 11.4 ± 0.5 |
| | 5 mg/ml F094 | 12.0 ± 1.3 |
| | 10 mg/ml F094 | 12.0 ± 0.4 |
| Prilocaine HCl | 0 mg/ml F094 | 0.47 ± 0.03 |
| | 1 mg/ml F094 | 0.43 ± 0.02 |
| | 5 mg/ml F094 | 0.51 ± 0.02 |
| | 10 mg/ml F094 | 0.50 ± 0.01 |
| Bupivicane HCl | 0 mg/ml F094 | 3.3 ± 0.4 |
| | 1 mg/ml F094 | 3.6 ± 0.5 |
| | 5 mg/ml F094 | 4.1 ± 0.3 |
| | 10 mg/ml F094 | 3.5 ± 0.8 |

Masses of Lidocaine HCl, Prilocaine HCl, and Bupivacaine HCl were measured by HPLC.
Values are the mean partition coefficient, plus or minus the standard deviation. These values are not statistically different from one another as shown by ANOVA.

All of the composition and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the claims.

### X. REFERENCES

The following references, to the extent that they provide exemplary procedural or other details supplementary to those set forth herein, are specifically incorporated herein by reference.
U.S. Pat. No. 3,070,623
U.S. Pat. No. 3,070,624
U.S. Pat. No. 5,049,388
U.S. Pat. No. 5,607,915
U.S. Pat. No. 6,444,233
Alfa et al., In: Experiments with Fission Yeast: A Laboratory Course Manual, Cold Spring Harbor Laboratory Press, Cold Spring Harbor, N.Y., 1993.
Beere and Green, Trends Cell Biol., 11:6-10, 2001.
Beere et al., Nat. Cell Biol., 2:469-475, 2000.
Belgian Patent 753773
Benhar et al., EMBO Rep., 3:420-425, 2002.
Boucaud et al., J Control Release, 81: 113-9, 2002.
British Patent 1346871
Chauhan et al., Blood, 103:3158-3166, 2004.
Creagh et al., Exp. Cell Res., 257:58-66, 2000.
Creagh et al., Leukemia, 14:1161-1173, 2000.
Ditzel et al., Nat. Cell Biol., 5:467-473, 2003.
Doll, et al., Lancet., 1:793, 1962.
Fang et al., Semin. Cancer Biol., 13:5-14, 2003.
Feder and Hofmann, Annu. Rev. Physiol., 61:243-282, 1999.
Finnin et al., J. Pharm. Sci., 88: 955-8. 1999.
Firestein and Feuerstein, J. Biol. Chem., 273:5892-5902, 1998.
Fnley et al., Cell, 48:1035-1046, 1987.
Gutterman et al., Proc. Natl. Acad. Sci. USA, 102: 12771-6, 2005.
Hanausek et al., Proc. Natl. Acad. Sci. USA, 98:11551-11556, 2001.
Haridas et al., J. Clin. Invest., 113:65-73, 2004.
Haridas et al., Proc. Natl. Acad. Sci. USA, 98:11557-11562, 2001.
Haridas et al., Proc. Natl. Acad. Sci. USA, 98:11557-11562, 2001.
Haridas et al., Proc. Natl. Acad. Sci. USA, 98:5821-5826, 2001.
Hershko et al., Annu. Rev. Biochem., 67:425-479, 1998.
Hideshima et al., cancer Res., 61:3071-3076, 2001.
Holcik et al., Apoptosis, 6:253-261, 2001.
Hostettmann et al., In: Saponins, Cambridge University Press, 1-548, 1995.
Huang et al., Zhongueo Yaoii Xuebao, Chem. Abstract No. 98100885, 3:286-288, 1982.
Inoue et al., Chem. Pharm. Bull., 6(2):897-901, 1986.
Japanese Pub. JP-A-44-32798
Jayatilake et al., J. Nat. Prod., 66:779-83, 2003.
Jesenberger and Jentsch, Nat. Rev. Mol. Cell Biol., 3:112-121, 2002.
Jesenberger and Jentsch, Nat. Rev. Mol. Cell Biol., 3:112-121, 2002.
Jolly and morimoto, J. Natl. Cancer Inst., 92:1564-1572, 2000.
Karande et al., Nat. Biotechnol., 22: 192, 2004.
Kasiwada et al., J. Org. Chem., 57:6946-6953, 1992.
Kisselev and Goldberg, Chem. Biol., 8:739-758, 2001.
Koepp et al., Cell, 97:431-434, 1999.
Kong et al., Phytochemistry, 33:427-430, 1993.
Lee and Goldberg, Mol. Cell. Biol., 18:30-38, 1998.
Mahato & Sen, Phytochemistry, 44:1185-1236, 1997.
McClintock, Science, 226:792-801, 1984.
Meng et al., Proc. Natl. Acad. Sci. USA, 96:10403-10408, 1999.
Mitsiades et al., Proc. Natl. Acad. Sci. USA, 99:14374-14379, 2002.
Mujoo et al., Cancer Res., 61:5486-5490, 2001.
Mujoo et al., Cancer Res., 61:5486-5490, 2001.
Mujoo et al., Oncogene, 12:1617-1623, 1996.
Myung et al., Med. Res. Rev., 21(4):245-273, 2001.
Nagamoto et al., Planta Medica., 54:305-307, 1988.
Nylandsted et al., Proc. Natl. Acad. Sci. USA, 97:7871-7876, 2000.
Pagano et al., Science, 269:682-685, 1995.
Palombella et al Proc. Natl. Acad. Sci. USA, 95:15671-15676, 1998.
Prausnitz, et al., Proc. Natl. Acad. Sci. USA, 90: 10504-8, 1993.
Purdon et al., Crit. Rev. Ther. Drug. Carrier Syst., 21: 97-132, 2004.
Pisha et al., Nature Medicine, 1:1046-1051, 1995.
Ravagnan et al., Natl. Cell Biol., 3:839-843, 2001.
Remington's Pharmaceutical Sciences 15th Edition, 33:624-652, 1990
Remington's Pharmaceutical Sciences, 16th Ed. Mack Publishing Company, 1980
Rose et al., In: Methods in Yeast Genetics: A Laboratory Course Manual, Cold Spring Harbor Laboratory, Cold Spring Harbor, N.Y., 1990.
Saleh et al., Nat. Cell Biol., 2:476-483, 2000.
Sambrook et al., In: Molecular Cloning:

A Laboratory Manual, Cold Spring Harbor Laboratory Press, NY, Vol 1, 2, 3, 1989.
Sarge et al., Mol. Cell. Biol., 13:1392-1407, 1993.
Schimmer et al., Cancer Cell, 5:25-35, 2004.
Sun et al., Mol. Cell, 14:81-93, 2004.
Suzuki et al., J. Biol. Chem., 276: 27058-27063, 2001.
Tamm et al., Clin. Cancer Res., 6:1796-1803, 2000.
Thompson et al., Cancer Epidemiol. Biomarker Prevent., 1:597-602, 1992.
Tomas-Barbaren et al., Planta Medica., 54:266-267, 1988.
Varshaysky, Nat. Cell Biol., 5:373-376, 2003.
Weissman, Nat. Rev. Mol. Cell Biol., 2:169-178, 2001.
Wendt et al. Agnew. Chem. Int. Ed. Engl., 39:2812-2833, 2000.
Wyllie, Anticancer Res., 5:131-136, 1985.
Yamada et al., J. Cell Sci., 110:1793-1804, 1997.
Yang and Yu, FASEB J., 17:790-799, 2003.
Yen et al., J. Biol. Chem., 278:30669-30676, 2003.
Boucaud et al., J. Control Release 81(1-2): 113-9, 2002.
Finnin et al., J. Pharm. Sci. 88(10): 955-8, 1999.
Gutterman et al., Proc. Nat'l Acad. Sci. USA 102(36): 12771-6, 2005.
Haridas et al., J. Clin. Invest. 113(1): 65-73, 2004.
Haridas et al., Proc. Nat'l Acad. Sci. USA 98(10): 5821-6, 2001.
Haridas et al., Proc. Nat'l Acad. Sci. USA 102(29): 10088-93, 2005.
Jayatilake et al., J. Nat. Prod. 66(6): 779-83, 2003.
Karande et al., Nat. Biotechnol. 22(2): 192, 2004
Mujoo et al., Cancer Res. 61(14): 5486-90, 2001.
Prausnitz et al., Proc. Nat'l Acad. Sci. USA 90(22): 10504-8, 1993.
Purdon et al., Crit. Rev. Ther. Drug Carrier Syst. 21(2): 97-132, 2004.

1. A pharmaceutical composition comprising (i) an avicin, and (ii) a therapeutic or diagnostic agent, wherein said composition is formulated for topical, percutaneous, transmucosal, transepithelial, transendothelial or transdermal administration.

2. The composition of claim 1, wherein said avicin is Avicin D and/or Avicin G.

3. The composition of claim 2, wherein said composition is essentially free of avicins other than Avicin D and/or Avicin G.

4. The composition of claim 2, wherein said composition comprises at least one avicin in addition to Avicin D and Avicin G.

5. The composition of claim 4, wherein said composition comprises F094.

6. The composition of claim 1, wherein said therapeutic agent is an anti-cancer agent.

7. The composition of claim 6, wherein said anti-cancer agent is a chemotherapeutic, a radioisotope, a toxin, a hormone, a cell-cycle regulator, a tumor suppressor, an anti-angiogenic agent, a gene silencing agent, or a pro-apoptotic protein.

8. The composition of claim 1, wherein said therapeutic agent is an agent other than an anti-cancer agent.

9. The composition of claim 8, wherein said non-anti-cancer therapeutic agent is an antibiotic, an antiviral, an anti-inflammatory, a cardiovascular drug, a cosmeceutical, an anesthetic, a toxin, an anti-coagulant or a hormone.

10. The composition of claim 8, wherein said non-anti-cancer therapeutic agent is a water-soluble anesthetic.

11. The composition of claim 8, wherein said non-anti-cancer therapeutic agent is a lidocaine or a prilocaine or a bupivacaine.

12. The composition of claim 1, wherein said diagnostic agent is a peptide, a protein or small molecule.

13. The composition of claim 1, wherein said therapeutic or diagnostic agent is hydrophilic.

14. The composition of claim 1, wherein said therapeutic or diagnostic agent is hydrophobic.

15. The composition of claim 1, wherein said composition is formulated for topical administration.

16. The composition of claim 15, wherein the topical formulation is a cream, ointment, salve, spray, gel, lotion, emulsion, powder, liquid aerosol, or powder aerosol.

17. The composition of claim 1, wherein said composition is formulated for transmucosal, transepithelial, transendothelial or transdermal administration.

18. The composition of claim 17, wherein the transdermal formulation is a patch.

19. The composition of claim 1, further comprising a chemical penetration enhancer, a membrane permeability agent, a membrane transport agent, a preservative, a surfactant or a stabilizer.

20. A pharmaceutical composition comprising purified Avicin G free of other avicins, wherein said composition is formulated topical or transdermal administration.

21. The composition of claim 20, further comprising a cosmeceutical agent.

22. The composition of claim 20, wherein said composition is a cream, gel, salve, ointment, lotion, powder, liquid aerosol, powder aerosol, emulsion or spray.

23. A method of promoting percutaneous, transmucosal, transepithelial, transendothelial or transdermal transport of a therapeutic or diagnostic agent in an animal subject comprising applying a pharmaceutical composition in accordance with claim 1 comprising an avicin preparation and said agent to body surface.

24-43. (canceled)

44. A method of altering water retention by a tissue comprising (a) identifying a human subject in need of water retention alteration, and (b) applying a pharmaceutical formulation comprising an avicin to a tissue surface.
